(19) United States
(12) Patent Application Publication
(10) Pub. No.: US 2026/0369407 A1
(43) Pub. Date: Nov. 19, 2026

(54) GenAI-Powered Intelligent Prior Authorization Assistant

(71) Applicant: Cerner Innovation, Inc., Kansas City, MO (US)

(72) Inventors: Suman Pal, Bangalore (IN); Pragnya Ranjan Pradhan, Similipada (IN); Chetan KV, Bangalore (IN); Michael Chung Kun Chen, San Jose, CA (US); Oshin Benny Anto, Kerala (IN); Shanthan Kesharaju, Cary, NC (US); Ameen Tayyebi, Miami, FL (US)

(73) Assignee: Cerner Innovation, Inc., Kansas City, MO (US)

(21) Appl. No.: 19/389,251

(22) Filed: Nov. 14, 2025

Related U.S. Application Data
(60) Provisional application No. 63/804,799, filed on May 13, 2025.

(51) Int. Cl. G16H 50/20; G16H 10/60

(52) U.S. Cl. CPC G16H 50/20; G16H 10/60

(57) ABSTRACT

Techniques for leveraging GenAI to (a) extract and parse unstructured data for use by GenAI to automatically generate evaluations of medical necessity for medical procedures for patient conditions, (b) automatically generate questionnaires for use by GenAI to automatically evaluate medical necessity for medical procedures for patient conditions, and (c) automatically generate prefilled questionnaires for use by GenAI in evaluating medical necessity of medical procedures for patient conditions are disclosed. The system may apply a first GenAI model to a clinical guideline to generate a questionnaire and clinical guideline rules. The system may apply a second GenAI model to the questionnaire and patient data to generate a prefilled questionnaire. The system applies a third GenAI model to the prefilled questionnaire and the clinical rules to determine medical necessity of the medical procedure. The system generates a prior authorization request based on the determination of medical necessity.

### BENEFIT CLAIMS; RELATED APPLICATIONS; INCORPORATION BY REFERENCE

This application claims the benefit of U.S. Provisional Patent Application 63/804,799, filed May 13, 2025, which is hereby incorporated by reference.

The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to machine learning (ML) applications in automated healthcare systems. In particular, the present disclosure relates to the application of generative artificial intelligence (GenAI) in prior authorization workflows.

### BACKGROUND

Prior authorization (PA) is a healthcare process requiring patients or their providers to secure approval from the health insurance company before proceeding with a specific treatment, medication, or procedure. Prior authorization ensures the service is covered by insurance and meets the insurer’s medical necessity criteria.

To streamline and standardize prior authorization, the Da Vinci Project (a Health Level 7 Fast Healthcare Interoperability Resources (HL7 FHIR) accelerator) introduced three key standards: Coverage Requirements Discovery (CRD), Documentation Templates and Rules (DTR), and Prior Authorization Support (PAS). CRD identifies if PA is needed, integrates with electronic health records (EHRs), and reduces unnecessary requests. DTR collects required clinical documentation, ensuring compliance and minimizing rework. PAS automates prior authorization submission and responses via FHIR application programming interfaces (APIs).

In accordance with the Da Vinci Project, the prior authorization workflow begins when a provider initiates a patient encounter and decides to order a service, medication, or procedure. Updates to EHR trigger Clinical Decision Support (CDS) Hooks. The EHR system sends patient, provider, and service details to the payer’s CRD service. The payer system evaluates what prior authorization is required, what documentation is needed, and any alternative covered options. The EHR system receives the CRD results and displays a decision card. The provider uses DTR to fetch payer-specific templates. The provider completes required documentation, and Clinical Quality Language (CQL) is used to auto-populate data from the EHR, where applicable. Completed documentation is submitted via PAS, allowing the payer to evaluate the prior authorization request. The payer system then sends an authorization response to the EHR system. The authorization decision is displayed in the EHR. If approved, the provider proceeds with the care. If denied, the provider addresses the issue.

Automating prior authorization in healthcare faces significant technical challenges despite being a high-value use case for interoperability and artificial intelligence (AI) applications. Healthcare data is fragmented across disparate EHR systems, payer platforms, and pharmacy systems, many of which utilize different data standards or rely on unstructured formats, such as free-text physician notes or scanned documents. Even where interoperability standards exist, adoption is inconsistent and incomplete.

Other challenges arise from rule heterogeneity and system integration. Different entities may maintain their own authorization rules, forms, and criteria, which are subject to frequent change and may not be published in machine-readable formats. Thus, automated systems are required to be able to adapt to opaque and dynamic rules. As a result, any hard-coding of such rules into software systems is required to be repeatedly reconfigured or patched, which may create software instability, frequent downtime, and inconsistent execution of rule-based logic across systems. Poorly designed prior authorization automation may also disrupt workflows, introduce redundant alerts, and/or cause increased system inefficiencies instead of reductions.

The approaches described in this section are approaches that could be pursued but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a prior authorization decision support system in accordance with one or more embodiments;

FIG. 2A illustrates an example set of operations for applying GenAI to structured and unstructured inputs for use by GenAI to automatically generate evaluations of medical necessity for medical procedures for patient conditions in accordance with one or more embodiments;

FIG. 2B illustrates an example set of operations for applying GenAI to automatically generate questionnaires for use by GenAI in evaluating medical necessity of medical procedures for patient conditions in accordance with one or more embodiments;

FIG. 2C illustrates an example set of operations for applying GenAI to automatically generate prefilled questionnaires for use by GenAI in evaluating medical necessity of medical procedures for patient conditions in accordance with one or more embodiments;

FIG. 3A illustrates an example clinical guideline in accordance with one or more embodiments;

FIG. 3B illustrates an example clinical guideline questionnaire in accordance with one or more embodiments;

FIG. 3C illustrates an example of a prefilled questionnaire in accordance with one or more embodiments;

FIG. 3D illustrates an example of an evaluation with reasoning of medical necessity of a medical procedure in accordance with one or more embodiments;

FIG. 3E illustrates an example prior authorization request for a medical procedure in accordance with one or more embodiments;

FIG. 4A illustrates a machine learning (ML) engine in accordance with one or more embodiments;

FIG. 4B illustrates an example set of operations of an ML engine in accordance with one or more embodiments; and

FIG. 5 shows a block diagram that illustrates a computer system in accordance with one or more embodiments.

### DETAILED DESCRIPTION

1. GENERAL OVERVIEW
2. PRIOR AUTHORIZATION DECISION SUPPORT SYSTEM ARCHITECTURE
3. LEVERAGING GENAI TO EXTRACT AND PARSE STRUCTURED AND UNSTRUCTURED DATA FOR USE BY GENAI TO AUTOMATICALLY GENERATE EVALUATIONS OF MEDICAL NECESSITY FOR MEDICAL PROCEDURES FOR PATIENT CONDITIONS
4. LEVERAGING GENAI TO GENERATE QUESTIONNAIRES FOR USE BY GENAI IN EVALUATING MEDICAL NECESSITY OF MEDICAL PROCEDURES FOR PATIENT CONDITIONS
5. LEVERAGING GENAI TO GENERATE PREFILLED QUESTIONNAIRES FROM UNSTRUCTURED DATA FOR USE BY GENAI IN EVALUATING MEDICAL NECESSITY OF MEDICAL PROCEDURES FOR PATIENT CONDITIONS
6. EXAMPLE EMBODIMENTS
7. MACHINE LEARNING ARCHITECTURE
8. MACHINE LEARNING ENGINE OPERATIONS
9. GENERATIVE AI MODELS
10. PRACTICAL APPLICATIONS, ADVANTAGES & IMPROVEMENTS
11. HARDWARE OVERVIEW
12. MISCELLANEOUS; EXTENSIONS

### 1. General Overview

One or more embodiments leverage GenAI to (a) extract and parse unstructured data for use by GenAI to automatically generate evaluations of medical necessity for medical procedures for patient conditions, (b) automatically generate questionnaires for use by GenAI to automatically evaluate medical necessity for medical procedures for patient conditions, and (c) automatically generate prefilled questionnaires for use by GenAI to evaluate medical necessity of medical procedures for patient conditions. Initially, the system accesses clinical guidelines (e.g., Medicare National Coverage Determination (NCDs) and Local Coverage Determination (LCDs)) and payer specific policies. The system prompts a first GenAI model (e.g., large language model (LLM)) to generate structured questionnaires and clinical guideline rules from clinical guidelines. The system applies the prompted first GenAI model to the clinical guidelines to generate questionnaires and clinical guideline rules. The questionnaires and clinical guideline rules align with insurer requirements and improve consistency in evaluating medical necessity of medical procedures.

One or more embodiments extract and parse structured and unstructured data (e.g., patient data) from EHRs to provide to a second GenAI model to populate a relevant questionnaire. Patient records contain vast amounts of information, including free text, making manual extraction of relevant data tedious and time-consuming. The system parses the patient data into subsets of patient data. Using one or more ML models, the system analyzes the subsets of the patient data to determine relevant subsets of data for answering each question of the questionnaire. The system applies the second GenAI model to the relevant subsets of patient data to determine one or more particular subsets of patient data to answer each question of the questionnaire. The system may generate explanations and citations for particular questions directly from the patient data. The application of ML to automatically extract relevant patient data from an EHR and prefill questionnaires for evaluating medical necessity reduces manual input errors and ensures completeness.

One or more embodiments determine medical necessity of a medical procedure for a patient condition using the clinical guideline rules generated by the prompted first GenAI model and the questionnaire generated by the prompted first GenAI model and populated by the second prompted GenAI model. More particularly, the system uses a prompted third GenAI model, e.g., LLM, applied to the clinical guideline rules and prefilled questionnaire to generate an evaluation that indicates if the medical procedure for the patient condition is medically necessary. The system may provide reasoning for determining medical necessity or no medical necessity, thereby improving decision making and reducing back-and-forth with third-party payers.

One or more embodiments described in this Specification and/or recited in the claims may not be included in this General Overview section.

### 2. Prior Authorization Decision Support System Architecture

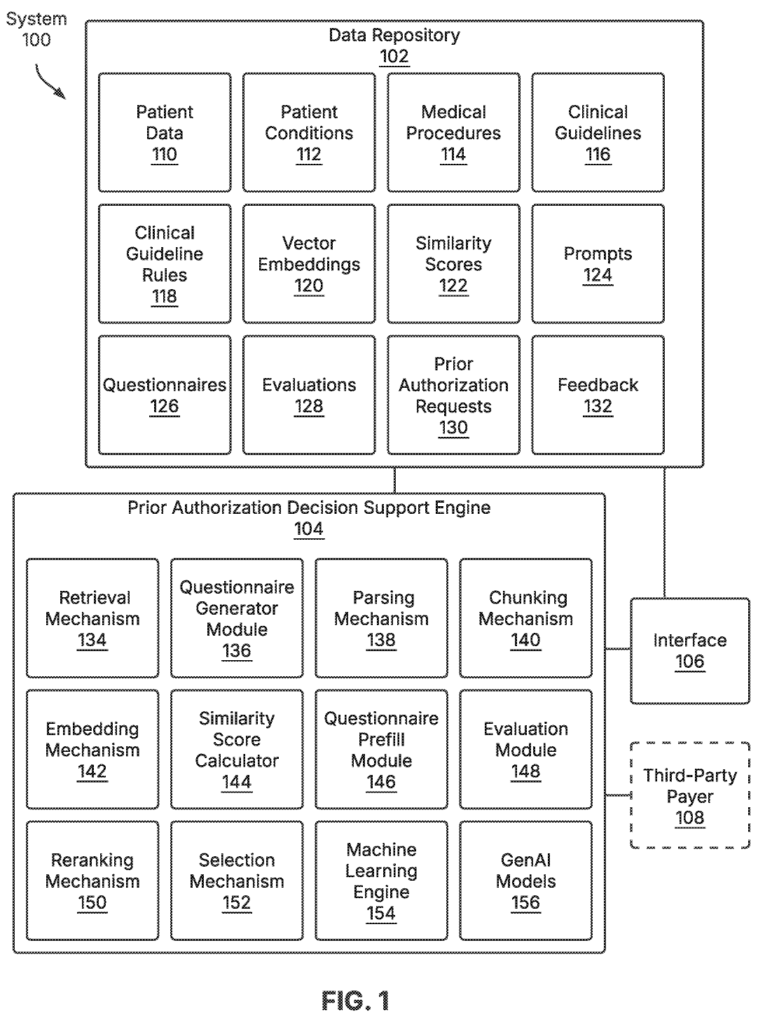

FIG. 1 illustrates a prior authorization decision system 100 in accordance with one or more embodiments. As illustrated in FIG. 1, system 100 includes a data repository 102, a prior authorization decision support engine 104, an interface 106, and optionally, a third-party payer 108. In one or more embodiments, system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, data repository 102 is any type of storage unit and/or device (e.g., a file system, database, collection of tables, or any other storage mechanism) for storing data. Furthermore, data repository 102 may include multiple different storage units and/or devices. The multiple different storage units and/or devices may or may not be of the same type or located at the same physical site. Furthermore, data repository 102 may be implemented or executed on the same computing system as prior authorization decision support engine 104. Additionally, or alternatively, a data repository 102 may be implemented or executed on a computing system separate from prior authorization decision support engine 104. Data repository 102 may be communicatively coupled to prior authorization decision support engine 104 via a direct connection or via a network.

Information describing leveraging GenAI to automatically generate prior authorization reports for medical procedure requests for patients may be implemented across any of components within the system 100. However, this information is illustrated within the data repository 102 for purposes of clarity and explanation.

In one or more embodiments, data repository 102 includes patient data 110, patient conditions 112, medical procedures 114, clinical guidelines 116, clinical guideline rules 118, vector embeddings 120, similarity scores 122, prompts 124, questionnaires 126, evaluations 128, prior authorization requests 130, and feedback 132.

In one or more embodiments, patient data 110 refers to structured, semi-structured, or unstructured information that characterizes an individual’s health status, medical history, and course of clinical care. Patient data 110 may include demographics and identifiers (e.g., name, date of birth, medical record number, insurance ID), medical history and problem lists, current and past diagnoses, medications, allergies, vital signs, laboratory results, imaging findings, procedures, and encounters. Patient data 110 may also include social and behavioral determinants of health, payer information, and patient-generated data from wearables or home monitoring devices.

In one or more embodiments, clinical notes include history and physical exams, progress notes, consultations, operative and discharge summaries, and/or secure messages. Clinical notes may include structured or unstructured text that identify problems, symptoms, physical findings, assessments/plans, procedures, medications, allergies, vitals, and social factors. Clinical notes may include terminologies, e.g., SNOMED CT, ICD-10-CM, CPT/HCPCS, and RxNorm.

In one or more embodiments, diagnostic results include laboratory and imaging data. Diagnostic results may include order or test identifiers, measured values and Unified Code for Units of measure (UCUM) units, reference ranges, abnormal flags, specimen and collection metadata, device identifiers, and/or interpretive statements. Observations associated with diagnostic results may include modality, body site, technique, structured findings, and impressions. Diagnostic results may be temporally indexed. Temporal indexing of diagnostic results may support longitudinal queries, e.g., the last three HBA1C values within 180 days.

In one or more embodiments, family medical history refers to a structured dataset that encodes health conditions observed in an individual’s biological relatives. In a family medical history, the system may represent a relative as a node with attributes comprising relationship type and degree (e.g., mother, father, sibling, second-degree), lineage side (maternal/paternal), biologic/adoptive status, twin/consanguinity flags, and vital status. The system may link each node to one or more condition records, including standardized codes (e.g., SNOMED CT/ICD-10-CM), age or period of onset, severity, outcome (e.g., cause of death), and/or genetic findings (e.g., gene/variant identifiers) and risk modifiers. Negative assertions (e.g., “no known family history of colorectal cancer”) may be captured explicitly with assertion type and observation date. The nodes may carry provenance (source document or intake form, clinician/author, collection method), timestamps, and extraction confidence, and nodes are normalized to interoperable resources (e.g., HL7 FHIR FamilyMemberHistory with linked Condition/Observation elements) to support deterministic queries. A reconciliation process may merge duplicate or conflicting reports across sources (EHR notes, patient-reported forms, genetic reports) using recency, source reliability, and concept equivalence rules.

In one or more embodiments, patient data 110 is stored across multiple data storage sources, including EHR systems, FHIR-compliant APIs, payer claims repositories, health information exchanges (HIE), laboratory and imaging systems, and connected patient portals or mobile applications. Prior authorization decision support engine 104 may employ an interoperability layer to access and unify these sources.

In one or more embodiments, patient conditions 112 refer to a clinically recognized state, disorder, disease, injury, and/or physiological status associated with a patient. Patient conditions 112 may be represented as a structured entity that captures a diagnosis or suspected diagnosis, clinical manifestation, symptom cluster, or health status indicator. Patient conditions 112 may include numerous attributes, such as standardized codes (e.g., SNOMED CT, ICD-10-CM, MedDRA, LOINC), onset date and duration, severity (e.g., mild, moderate, severe), acuity (e.g., acute, chronic, recurrent), stage or classification (e.g., TNM staging, NYHA class), episodicity, and prognosis.

In one or more embodiments, patient conditions 112 are linked to one or more clinical observations, diagnostic results, and/or treatment procedures. For example, a condition such as “Type 2 Diabetes Mellitus” may be associated with laboratory results (e.g., HbA1c values), medications (e.g., metformin), and complications (e.g., diabetic retinopathy). Patient conditions 112 may further include contextual modifiers, such as suspected vs. confirmed status, primary vs. secondary diagnosis, or active vs. resolved condition. Patient conditions 112 may carry provenance metadata, including source document or system (e.g., provider note, claims data, registry record), authoring clinician, method of capture (manual entry, NLP extraction, FHIR Condition resource ingestion), and timestamp information. Conditions may be normalized to interoperable resources (e.g., HL7 FHIR Condition resource linked to Observation, Procedure, or Encounter elements) to support deterministic queries and decision support. Negative assertions may also be captured explicitly within patient conditions 112 (e.g., “no history of myocardial infarction”) with assertion type, observation date, and source. A reconciliation process may be applied to merge duplicate or conflicting condition entries across disparate sources using recency, data source reliability, concept normalization, and confidence scoring rules.

In one or more embodiments, medical procedures114 refer to a set of diagnostic, therapeutic, preventive, and/or surgical interventions performed on a patient to evaluate, manage, and/or treat a health condition. Medical procedures 114 may be represented as structured records keyed by time, encounter, ordering provider, and/or performing facility. Medical procedures 114 may include standardized identifiers and codes, e.g., CPT, HCPCS, ICD-10-PCS, LOINC, SNOMED CT, and/or proprietary payer codes.

In one or more embodiments, each medical procedure record may include various attributes, such as procedure type (e.g., surgical, imaging, laboratory, interventional, rehabilitative), intent (diagnostic, therapeutic, palliative, preventive), procedure description, procedure status (scheduled, in-progress, completed, cancelled), performing provider or team, facility location, equipment/device identifiers, and anesthetic or sedation details. Medical procedures 114 may include clinical indications, patient preparation requirements, procedural techniques, and associated risks or complications. Metadata associated with medical procedures 114 may comprise timestamps (order date, start time, end time), procedure priority or urgency flags (routine, urgent, emergent), order provenance (ordering provider, order system, referral), and authorization details (payer approval identifiers, prior authorization status, coverage determinations). Medical procedures 114 may be temporally indexed to enable longitudinal analyses (e.g., identifying repeated interventions, tracking outcomes of staged procedures, or correlating with diagnostic timelines).

In one or more embodiments, medical procedures 114 may be linked to related clinical entities, including patient conditions (e.g., indication for the procedure), diagnostic results (e.g., imaging studies, pathology reports), medications (e.g., peri-procedural drugs), and outcomes (e.g., adverse events, complications, or follow-up requirements). Normalization of medical procedure data to interoperable resources (e.g., HL7 FHIR Procedure resource linked with Condition, Observation, and Encounter resources) supports deterministic queries, cross-system interoperability, and decision support. Negative or null procedures may also be represented, such as documentation that a specific procedure was considered but not performed, deferred, or contraindicated. Reconciliation processes may merge duplicate or conflicting procedure records across heterogeneous sources (EHR systems, claims data, registries) using rules for recency, source reliability, and standardized code equivalence.

In one or more embodiments, clinical guidelines 116, also known as payer medical policies, coverage criteria, or utilization-management guidelines, are guidelines created by health insurance providers, e.g., insurance companies, for determining whether or not to authorize a medical procedure for a patient, e.g., an insured individual. Clinical guidelines 116 may be particular to an insurance provider, organization, or governing body. Clinical guidelines 116 for the same insurer may vary based on geography, e.g., from state to state and/or from country to country. Clinical guidelines provided by the Centers for Medicare & Medicaid Services (CMS) include NCD and LCD. NCDs are clinical guidelines that provide for decisions made at the national level, and they apply to Medicare beneficiaries across the United States. LCDs provide for decisions made by Medicare Administrative Contractors that administer Medicare claims and benefits in specific geographic regions or jurisdictions.

In one or more embodiments, clinical guidelines 116 are particular to an insurance provider. Clinical guidelines 116 may include eligibility criteria, approved treatments, required documentation, treatment protocols, and exclusions. Clinical guidelines 116 may be accessed through insurance company websites and provider portals. Insurance companies may provide clinical policy information and coverage guidelines on their websites. Web scraping may be implemented to access the information from the websites. Insurance providers may provide dedicated portals for accessing clinical guidelines and coverage criteria. Insurance providers may offer APIs that enable secure access to databases including clinical guidelines and coverage information.

In one or more embodiments, clinical guideline rules 118 refer to structured, machine-interpretable representations of evidence-based clinical criteria that define the conditions under which a medical procedure is considered medically necessary, appropriate, or reimbursable. Clinical guideline rules 118 operationalize the textual content of published medical guidelines, payer coverage determinations, or clinical policy bulletins into executable logic suitable for automated evaluation by a prior authorization decision support engine 104.

In one or more embodiments, a clinical guideline rule includes one or more logical statements or constraints that specify qualifying conditions (e.g., required diagnoses, age, laboratory values, prior treatments, contraindications). Clinical guideline rules 118 may include Boolean operators (AND, OR, NOT), conditional branches, temporal dependencies (e.g., treatment within the last 90 days), or quantifiable thresholds linked to threshold scores. Clinical guideline rules 118 may be expressed in standardized or semi-structured formats, such as JSON, XML, or HL7 FHIR PlanDefinition/Library resources, enabling interoperability, version control, and/or automated execution. Each clinical guideline rule may carry identifiers (rule ID, guideline reference ID, version number), provenance metadata (source organization, publication date, review date), and jurisdictional applicability (payer, region, or clinical domain). Metadata associated with clinical guideline rules 118 may include evidence level indicators (e.g., A/B/C grade recommendations), rule dependencies, linked references (e.g., NCCN, CG-MED, CMS LCD/NCD), and outcome mappings (e.g., “medically necessary,” “not medically necessary,” “requires manual review”). These metadata attributes support rule transparency, auditability, and traceability across payer adjudication systems.

In one or more embodiments, vector embeddings 120 refer to multi-dimensional numerical representations of clinical, procedural, or administrative entities (e.g., patient data 110, patient conditions 112, medical procedures 114). Vector embeddings 120 may be generated by applying a vectorization function or ML model to structured and/or unstructured inputs. Vector embeddings 120 capture latent semantic, syntactic, and contextual relationships between medical concepts, enabling computational similarity, clustering, and classification operations. Vector embeddings 120 may represent (i) subsets of patient data and/or (ii) individual questions from clinical guideline questionnaires.

In one or more embodiments, vector embeddings 120 are generated by one or more AI models, including transformer-based large language models (LLMs), domain-specific encoders (e.g., Self-Alignment Pretraining for Biomedical Entity Representations (SapBERT), BioBERT, PubMedBERT), or multimodal embedding models that jointly encode text, imaging, genomic, and/or claims data. Vector embeddings 120 may be computed in fixed-length or variable-length dimensions (e.g., 128-D, 512-D, 1024-D) with each dimension representing a latent feature learned during model training. Vector embeddings 120 may include metadata comprising embedding type (e.g., diagnosis embedding, procedure embedding, note embedding), embedding function version, training corpus provenance, dimensionality, normalization method (e.g., L2 norm), and associated confidence scores. Embeddings may be indexed temporally to support longitudinal tracking (e.g., evolution of patient condition embeddings across encounters).

In one or more embodiments, vector embeddings 120 support different operations, including similarity computation (e.g., cosine similarity, Euclidean distance), clustering (e.g., k-means, hierarchical clustering), classification (e.g., nearest neighbor classification of diagnoses), ranking (e.g., retrieval of candidate procedures by semantic similarity), and threshold-based matching against threshold scores. Vector embeddings 120 may be persisted in a vector database or index structure optimized for approximate nearest neighbor (ANN) search (e.g., FAISS, ScaNN, Milvus, Pinecone). Vector embeddings 120 may be linked to corresponding structured entities (e.g., HL7 FHIR Condition, Procedure, or Observation resources) to support deterministic retrieval, auditability, and cross-model interoperability. Adaptive refinement may be applied to vector embeddings 120 through fine-tuning or continuous learning, wherein embeddings are recalibrated using updated guideline data, payer rules, or patient population distributions. Provenance metadata may capture training iterations, data versioning, and embedding lineage for compliance, reproducibility, and explainability.

In one or more embodiments, similarity scores 122 refer to quantitative indicators that measure the degree of correspondence between two or more vector embeddings 120. Similarity scores 122 may be expressed as normalized values (e.g., ranging from 0.0 to 1.0), percentage matches, or distance metrics inversely proportional to similarity. Similarity scores 122 may be computed by mathematical or statistical functions, including cosine similarity, dot product, Euclidean distance, Manhattan distance, or Kullback-Leibler divergence. Similarity scores 122 may represent model-derived confidence probabilities, ranking functions, or alignment weights within transformer architectures.

In one or more embodiments, similarity scores 122 are used to identify clinically relevant relationships such as a match between a subset of patient data 110 and a particular question from a clinical guideline questionnaire. Similarity scores may include metadata, comprising scoring method, normalization parameters, embedding dimensionality, model version, calculation timestamp, and provenance of input data. Additional contextual attributes may include patient identifiers, encounter metadata, or procedural categories to support stratified queries. Similarity scores 122 may further be applied in ranking operations, where candidate entities are ordered by descending similarity to a target, enabling top-k retrieval, reranking of generative AI outputs, or prioritization of procedures in a prior authorization workflow. Longitudinal analysis of similarity scores 122 may also support detection of clinical progression, shifts in coding practices, or evolving guideline adherence.

In one or more embodiments, similarity scores 122 may be evaluated against threshold scores to determine if a subset of patient data sufficiently matches a particular question from a clinical guideline questionnaire to consider the subset of patient data in an answer to the particular question. Threshold scores refer to quantitative or qualitative boundary values used to determine if a clinical, diagnostic, or procedural criterion has been satisfied. Threshold scores may be applied in the context of evaluating medical necessity, risk adjustment, diagnostic accuracy, treatment eligibility, or payer coverage criteria. Threshold scores may include static values predefined by clinical guidelines, payer policy, or regulatory standards or dynamic values learned or adapted through ML models.

In one or more embodiments, prompts124 refer to structured input instructions, queries, or contextual signals provided to a GenAI model to condition, guide, or constrain model outputs. Prompts 124 may be represented as natural language text, structured tokens, embeddings, or multimodal inputs (e.g., text with imaging metadata or patient data vectors). Prompts 124 may include one or more components, such as (i) task directives (e.g., “Evaluate medical necessity of the procedure based on guideline criteria.”); (ii) contextual patient information (e.g., demographics, diagnoses, recent diagnostic results); (iii) formatting instructions (e.g., structured JSON, FHIR-compliant response, ranked list); and/or (iv) constraints (e.g., limit to payer policy rules, enforce specific terminology).

In one or more embodiments, metadata associated with prompts 124 include prompt version, construction rules, originating module, timestamp, and/or linkage to input and output artifacts (e.g., vector embeddings 120, similarity scores 122). Prompts 124 may be logged with provenance attributes to support reproducibility, audit trails, and compliance monitoring. Prompts 124 may be optimized or refined through reinforcement learning, prompt engineering strategies (e.g., few-shot examples, chain-of-thought, retrieval-augmented generation (RAG), or adaptive feedback loops). Refined prompts may improve alignment of model outputs with clinical guidelines, payer rules, or decision-support objectives. Prompts 124 may support multi-turn interactions, wherein successive prompts incorporate feedback from prior outputs, reviewer annotations, or downstream decision outcomes. Multi-turn prompt flows may enable conversational review of medical necessity, iterative questionnaire generation, or explanation of authorization decisions.

In one or more embodiments, questionnaires 126 refer to structured collections of questions generated, curated, and/or instantiated to capture patient-specific or provider-supplied information relevant to the evaluation of medical necessity, clinical appropriateness, and/or payer authorization of medical procedures 114. Questionnaires 126 may be encoded in standardized formats (e.g., HL7 FHIR Questionnaire resource, XML, JSON) to support interoperability and automated processing. Questionnaires 126 may include multiple question types, such as binary (yes/no), categorical, Likert-scale, free-text, or conditional branching items. Each question may be associated with metadata, including question identifier, clinical concept linkage (e.g., SNOMED CT, LOINC, ICD-10-CM codes), display text, response options, validation rules, and branching logic. Adaptive branching logic may conditionally display subsequent questions based on prior responses, ensuring that the questionnaire remains targeted and context-specific.

In one or more embodiments, questionnaires 126 are dynamically generated. Metadata associated with questionnaires 126 may include version identifiers, provenance (e.g., system-generated, payer-supplied, clinician-authored), creation timestamp, and links to underlying guideline references or payer policies. Questionnaires 126 may carry audit attributes, such as source modules, model versioning for GenAI-driven construction, and usage logs for completed submissions. Questionnaires 126 may be stored as structured datasets (e.g., HL7 FHIR QuestionnaireResponse).

In one or more embodiments, questionnaires 126 include prefilled questionnaires. Prefilled questionnaires are questionnaires related to a medical procedure that has been populated with patient data. Prefilled questionnaires may be populated manually or automatically.

In one or more embodiments, evaluations 128 refer to structured assessments or determinations produced by applying computational models, decision rules, and/or hybrid AI-human review frameworks to patient data 110, patient conditions 112, medical procedures 114, and/or questionnaires 126. Evaluations 128 may represent an intermediate or final decision outcome, such as if a proposed procedure satisfies medical necessity, coverage criteria, or quality-of-care standards. Evaluations 128 may be expressed as categorical determinations (e.g., “approved,” “denied,” “requires additional information”), probabilistic confidence scores, or composite indexes that incorporate similarity scores 122, threshold scores, and/or vector embeddings 120. Evaluations 128 may include multiple dimensions, such as medical appropriateness, guideline compliance, cost-effectiveness, and patient-specific suitability. Metadata associated with evaluations 128 may include evaluation type (automated, semi-automated, manual), algorithm or model version, provenance of inputs (e.g., questionnaire 126 responses, diagnostic results, prompts 124), timestamp of evaluation, reviewer identity (if applicable), and linkage to governing clinical guidelines or payer policies.

In one or more embodiments, evaluations 128 may be generated through multi-stage pipelines where initial scoring models produce candidate outputs that are subsequently refined by generative AI reasoning modules, reranking layers, or human-in-the-loop verification. Evaluations 128 may further be reconciled across multiple sources (payer adjudication engines, provider-side decision support tools, or registry data) using recency, source reliability, and rules for conflict resolution. Evaluations 128 may be stored in interoperable formats (e.g., HL7 FHIR Task, CoverageEligibilityResponse, or custom adjudication artifacts) to support downstream workflows, such as payer authorization, provider notification, or quality reporting. Longitudinal analyses of evaluations 128 may support auditability, compliance monitoring, and performance benchmarking across populations and payer-provider ecosystems.

In one or more embodiments, prior authorization requests 130 refer to structured transactions submitted by a healthcare provider, payer system, and/or intermediary platform to obtain approval for a proposed medical procedure before the medical procedure is performed or reimbursed. Prior authorization requests 130 may be represented in standardized electronic formats (e.g., X12 278 Health Care Services Review, HL7 FHIR PriorAuthorization resource) or proprietary payer-defined schemas.

In one or more embodiments, prior authorization requests 130 include patient identifiers, demographics, encounter information, ordering provider details, requested service or procedure codes (e.g., CPT, HCPCS, ICD-10-PCS), associated diagnoses (ICD-10-CM, SNOMED CT), clinical documentation attachments (e.g., physician notes, lab results, imaging studies), and supporting questionnaire responses. Prior authorization requests 130 may include payer identifiers, coverage plan metadata, and authorization urgency flags (routine, urgent, emergent). Metadata associated with prior authorization requests 130 may include submission timestamp, request status (e.g., pending, approved, denied, pended for review), tracking identifiers, authorization reference numbers, and communication logs (provider-payer messages, attachments, follow-ups). Provenance information may capture originating system, submitting entity, and digital signatures or cryptographic hashes for audit compliance.

In one or more embodiments, prior authorization requests 130 are tracked longitudinally across payer-provider systems to support lifecycle management (submission, adjudication, appeal, renewal). Prior authorization requests 130 may be reconciled against claims data, EHR entries, and/or utilization review datasets to ensure consistency, detect fraud or errors, and support population-level reporting. Prior authorization requests 130 may be augmented with machine-readable provenance artifacts (e.g., blockchain-backed hashes, immutable audit trails) to enforce transparency, accountability, and/or regulatory compliance. Interoperable exchange of requests may be achieved through APIs, clearinghouses, or payer-provider networks adhering to standards such as HL7 Da Vinci Project specifications.

In one or more embodiments, feedback 132 refers to signals, annotations, or evaluative inputs provided by users, reviewers, and/or system components that indicate the accuracy, quality, and/or appropriateness of outputs generated within the prior authorization workflow. Feedback 132 may originate from clinicians, payers, patients, and/or automated adjudication engines and may include confirmations, corrections, approvals, denials, and/or requests for clarification.

In one or more embodiments, feedback 132 is structured or unstructured. Structured feedback may include coded outcomes (e.g., approval/denial codes, SNOMED CT assertions, reason-for-denial categories), numeric ratings (confidence scores, error severity levels), or Boolean indicators (true/false validation). Unstructured feedback may include free-text annotations, provider comments, or narrative justifications for authorization decisions. Feedback 132 may be used to refine similarity scores 122, threshold scores, and evaluations 128 by providing corrective and/or confirmatory inputs. Feedback 132 may trigger model retraining, parameter adjustment, and/or reinforcement learning loops for GenAI models 156.

Metadata associated with feedback 132 may include source (provider, payer, system), feedback type (manual review, automated rule engine result, human override), timestamp, provenance (EHR system, payer adjudication system, clinical portal), and linkage to the specific prompt, questionnaire, or evaluation being corrected or validated.

In one or more embodiments, feedback 132 is captured in interoperable formats (e.g., HL7 FHIR Communication, Task, or Provenance resources) to support traceability, audit trails, and compliance reporting. Longitudinal analysis of accumulated feedback may be used for bias detection, error trend analysis, and/or continuous improvement of clinical decision support systems. Feedback 132 may operate as machine-ingestible signals that drive adaptive system behavior, including the refinement of future prompts, restructuring of questionnaires, recalibration of threshold scores, and/or reranking of candidate outputs in authorization pipelines.

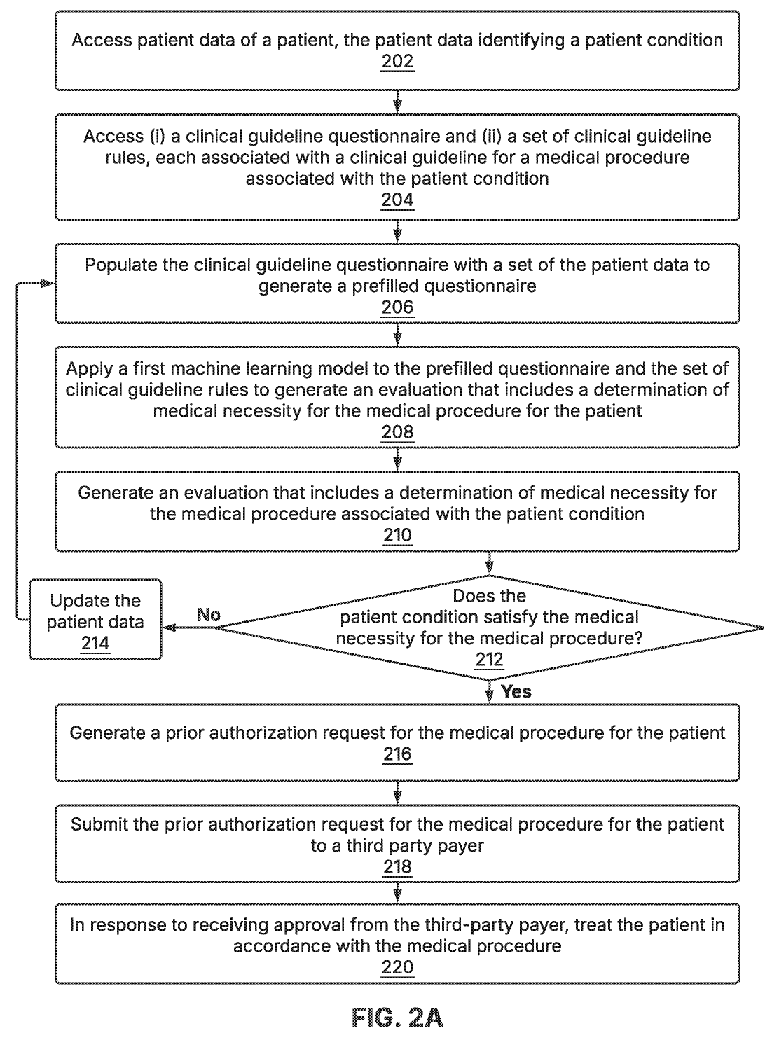

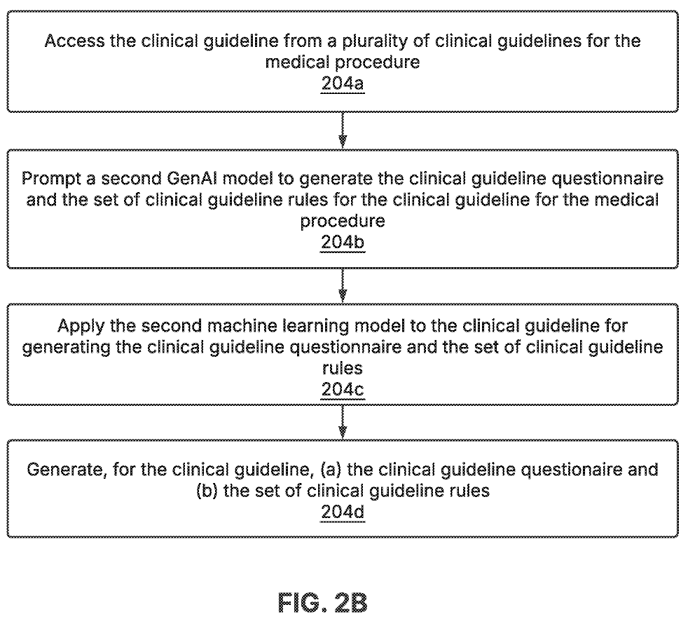

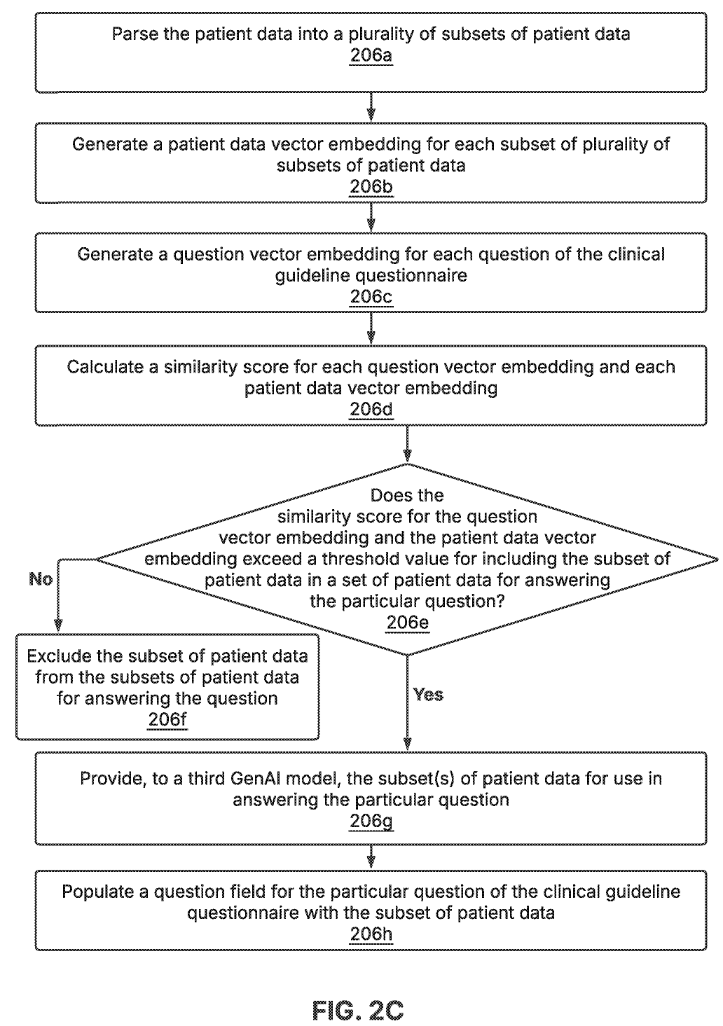

In one or more embodiments, prior authorization decision support engine 104 refers to hardware and/or software configured to perform operations described herein for leveraging GenAI to (a) automatically generate evaluations of the medical necessity of medical procedures for patients and (b) automatically generate prior authorization requests for medical procedures determined to be medically necessary. Examples are described below with reference to FIGS. 2A-2C.

In an embodiment, prior authorization decision support engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, prior authorization decision support engine 104 includes a retrieval mechanism 134. Retrieval mechanism 134 refers to hardware and/or software configured to perform operations described herein for accessing and retrieving information from data sources. This may include database retrieval and information retrieval. In relational databases, Structured Query Language (SQL) is used for querying and retrieving data. SQL allows for specifying exactly what data to retrieve and how it should be processed. In Not Only SQL (NoSQL) databases, retrieval mechanisms may vary significantly, depending on the type of NoSQL database, e.g., document, key-value, graph, or column store. These might use simpler, more scalable retrieval methods suited to the specific data model. Information retrieval is used primarily in search engines and large content management systems designed to handle unstructured data like text. Techniques include keyword search, semantic search, and full-text search. Retrieval mechanism 134 may use APIs to access data. Retrieval mechanism 134 may employ web scrapping techniques to acquire data from websites.

In one or more embodiments, prior authorization decision support engine 104 includes a questionnaire generator module 136. Questionnaire generator module 136 refers to hardware and/or software configured to perform operations described herein for generating structured questionnaires 126 and clinical guideline rules 118 for clinical guidelines 116. Questionnaire generator module 136 may operate in conjunction with one or more GenAI models 156 to synthesize, structure, and contextualize medical necessity criteria into machine- and human-interpretable question sets.

In one or more embodiments, questionnaire generator module 136 parses a selected clinical guideline or policy document (e.g., payer coverage guideline, CMS NCD/LCD, or medical policy) and identifies decision criteria, including qualifying diagnoses, demographic thresholds, prior treatment failures, contraindications, and outcome requirements. These criteria are transformed into discrete question templates. The question templates may be encoded with metadata, such as question identifiers, dependencies, required/optional flags, value types (Boolean, numeric, categorical, temporal), and response constraints. Questionnaire generator module 136 may employ a prompt-driven generative mechanism that applies a GenAI model to the parsed guideline text or rule representation. The GenAI model may translate narrative statements (e.g., “Procedure X is medically necessary when all of the following are met...”) into normalized question syntax (e.g., “Has the patient completed conservative medical management?”). Semantic alignment between rule text and question content may be established via vector embeddings 120 and similarity scores 122 to ensure that generated questions accurately reflect the intent of each clinical criterion.

In one or more embodiments, metadata associated with each generated questionnaire item may include guideline references (e.g., policy ID, section number, publication date), clinical concept bindings (e.g., SNOMED CT, ICD-10-CM, CPT/HCPCS, LOINC, RxNorm), and provenance attributes (e.g., originating model version, prompt configuration, timestamp, source guideline URI). Questionnaire generator module 136 may encode dependency structures defining logical operators (AND/OR) and branching conditions between questions to preserve hierarchical evaluation logic. Questionnaire generator module 136 may integrate with a questionnaire prefill module 146 to automatically populate question fields with relevant patient data 110 extracted from EHR or external sources. This integration enables dynamic, context-aware questionnaires that adapt to patient-specific attributes, thereby reducing manual clinician input and improving decision completeness.

In one or more embodiments, questionnaire generator module 136 produces the resulting questionnaire in machine-readable formats, such as HL7 FHIR Questionnaire resources, JSON, or XML. Generated questionnaires may be versioned, digitally signed, and stored with lineage metadata that links each question to the original clinical guideline rule and governing policy authority. Questionnaire generator module 136 may support rendering the questionnaire in human-readable form for clinical review or payer submission, ensuring semantic equivalence between machine and human representations. Questionnaire generator module 136 may include a feedback-driven refinement loop, wherein responses from reviewers, payers, and/or subsequent evaluations 128 are analyzed by GenAI or rule-based heuristics to refine future questionnaire generation. This allows the system to evolve with guideline updates, payer-specific requirements, and emerging best practices.

In one or more embodiments, prior authorization decision support engine 104 includes a parsing mechanism 138. Parsing mechanism 138 refers to hardware and/or software configured to perform operations described herein for ingesting, analyzing, and structurally normalizing patient data 110 obtained from disparate EHR systems and external clinical data repositories. Parsing mechanism 138 may accept heterogeneous clinical inputs, including structured messages (HL7 v2, FHIR JSON/XML), semi-structured documents (CCD, C-CDA, CSV, XLSX), unstructured narratives (clinical notes, discharge summaries, radiology reports, scanned documents), and imaging metadata (DICOM headers).

In one or more embodiments, parsing mechanism 138 applies natural language processing (NLP), optical character recognition (OCR), and entity normalization techniques to extract and standardize relevant clinical information. Extracted data may include demographics, encounter metadata, diagnoses, procedures, medications, allergies, vital signs, laboratory results, imaging findings, problem lists, and provider-authored notes. Parsing mechanism 138 may further include a sectionizer configured to detect canonical EHR sections (e.g., “History of Present Illness,” “Assessment and Plan,” “Medications,” “Allergies,” “Laboratory Results,” “Radiology Reports,” and “Discharge Summary”) and anchor each extracted entity to source document coordinates or timestamps.

In one or more embodiments, parsing mechanism 138 employs tokenization, pattern grammars (regular expressions, PEG/ANTLR rules), and/or ontology-assisted NLP to identify and extract various structured fields, such as (i) diagnoses and conditions mapped to standardized codes (ICD-10-CM, SNOMED CT); (ii) procedure and order entries (CPT, HCPCS, ICD-10-PCS); (iii) laboratory observations (LOINC, UCUM units, normal ranges, abnormal flags); (iv) medication orders (RxNorm, dosage forms, strength, route); and (v) encounter-level attributes (provider, location, specialty, care setting). Parsing mechanism 138 may normalize data units to UCUM standards, validate code syntax and referential integrity, resolve synonyms through controlled vocabularies (e.g., SNOMED CT, RxNorm, UMLS Metathesaurus), and reconcile duplicate or conflicting observations across multiple data sources using recency and source-reliability heuristics. The mechanism may also convert narrative clinical statements into executable predicates with parameterized expressions (e.g., threshold values, temporal windows, count limits, before/after relations), thereby enabling downstream computation of similarity scores 122 and evaluations 128.

In one or more embodiments, prior authorization decision support engine 104 includes a chunking mechanism 140. Chunking mechanism 140 refers to hardware and/or software configured to perform operations described herein for segmenting patient data 110 into machine-interpretable chunks suitable for retrieval, embedding, and deterministic evaluation. Chunking mechanism 140 may operate on structured, semi-structured, and unstructured clinical sources, including HL7/FHIR resources, CCD/C-CDA documents, CSV/XLSX datasets, and free-text provider notes extracted from EHRs, clinical registries, or payer data feeds. Chunking mechanism 140 may produce a sequence of versioned patient data chunk records. Each chunk record may include a stable chunk identifier, normalized text or data payload, structured metadata (e.g., encounter ID, observation type, time range, section/heading path, data modality), language and charset, token and character counts, boundary lineage (predecessor/successor chunk identifiers, source offsets), provenance (EHR system ID, document type, extraction timestamp, parser configuration/model version), and/or clinical context scope (encounter, episode, or longitudinal time window).

In one or more embodiments, chunking mechanism 140 employs multi-strategy segmentation, including (i) structural chunking, (ii) semantic chunking, and/or (iii) temporal chunking. Structural chunking honors native EHR hierarchies (e.g., Encounter → Section → Observation → Entry) and maintains logical containment of clinical facts (e.g., vital signs grouped with their measurement context). Semantic chunking uses topic-shift detectors, sectionizers, or transformer-based segmenters to align chunk boundaries with conceptual shifts (e.g., from diagnosis assessment to treatment plan). Temporal chunking partitions patient data into clinically meaningful time windows (e.g., most recent 90 days, per admission episode) to preserve context during evaluation. Chunking mechanism 140 may also enforce token-budget chunking to ensure each chunk fits the input size constraints of embedding and generative AI models with configurable overlap windows to maintain continuity across clinical events.

In one or more embodiments, chunking mechanism 140 normalizes whitespace, punctuation, dates, and units of measure to UCUM as well as resolves code systems to canonical identifiers (e.g., ICD-10-CM, LOINC, SNOMED CT, RxNorm). Duplicate or near-identical records may be deduplicated using k-shingling and content hashing across source systems. Adaptive chunking policies may dynamically tune chunk size and overlap based on data modality and density (e.g., smaller chunks for dense numerical data, such as labs, and larger chunks for narrative notes). Chunking mechanism 140 may employ hierarchical chunking to preserve parent-child relationships across EHR elements (e.g., problem list → condition → encounter), enabling semantic traversal across patient data layers. The resulting chunk graph facilitates downstream vector embedding generation, similarity computation, and cross-modal retrieval between patient data subsets and clinical guideline rules 118 for medical necessity evaluation.

In one or more embodiments, prior authorization decision support engine 104 includes an embedding mechanism 142. Embedding mechanism 142 performs one or more vector embedding functions. Vector embedding functions are mathematical functions that map objects, such as words, sentences, or other data points, into vector representations in a multi-dimensional space. These vector representations are used to capture the semantic or contextual meaning of the objects in a numerical format that can be easily processed by ML algorithms.

In some embodiments, the vector embedding functions are word embedding techniques. Word embedding techniques use NLP and ML to represent words as dense vectors of real numbers. Word embedding techniques aim to capture the semantic and syntactic meaning of words as well as their relationships with other words in a language. Word embedding techniques include Term Frequency-Inverse Document Frequency (TF-IDF), Word2Vec, Global Vectors (GLOVE), Large Language Models (LLMs), BioWordVec fastText, and Bidirectional Encoder Representation (BERT).

In one or more embodiments, the word embedding techniques include SAPBERT. The SAPBERT is a pre-trained BERT model trained in Medical Entity Linking (MEL) tasks. MEL maps various entities to unified concepts in the medical knowledge graph. Word representation learning faces a significant challenge due to the existence of heterogeneous names. For example, in healthcare, terms like ‘nostril’ and ‘nare’ are used interchangeably but yield considerably different embedding representations when generated by models not specifically trained for MEL. SAPBERT works on self-alignment of biomedical entity representation, so semantically similar entities belonging to the same concept are brought closer in the embedding space, forming compact clusters. SAPBERT leverages UMLS, the largest collection of biomedical concepts and synonyms, and collates the synonyms from various controlled vocabularies, e.g., SNOMED CT, MeSH, Gene Ontology, RxNorm, and OMIM. SAPBERT performs better compared to other variants of BERT, like Bio-BERT and Clinical-BERT, with respect to the MEL challenges. The SAPBERT model can accurately capture fine-grained semantic relationships and heterogeneous naming in the biomedical domain compared to other variants of BERT. The ability of SAPBERT to handle out-of-vocabulary terms, misspelled words, and rare medical terms provides a significant advantage over other models.

In one or more embodiments, embedding mechanism 142 hosts one or more encoder pipelines: (i) text encoders (e.g., transformer dual encoders) for narrative policy text, clinical notes, and plan documents; (ii) code-aware encoders that enrich text with standardized terminology features (ICD-10-CM/PCS, CPT/HCPCS, LOINC with UCUM units, RxNorm, SNOMED CT), including learned code embeddings and sparse one-hot or hash-bucket vectors; and/or (iii) structured-quantity encoders that serialize observations into canonical templates (e.g., “potassium 5.2 mmol/L on 2025-08-01”) after unit normalization to UCUM and then embed them or append compact numeric features (value z-scores, trend slopes) to the text vector.

In one or more embodiments, embedding mechanism 142 performs preprocessing (language/charset detection, text normalization, punctuation/whitespace harmonization, PHI redaction where required, and de-duplication via content hashes) and/or tokenization with corpus-specific rules (e.g., preserving code tokens like “73721” or “I10” as single units). Embedding mechanism 142 may support multilingual corpora and domain adapters, mean/CLS pooling, and optional L2 normalization to enable cosine similarity.

In one or more embodiments, prior authorization decision support engine 104 includes a similarity score calculator 144. Similarity score calculator 144 refers to hardware and/or software configured to perform the operations described herein for computing, normalizing, and/or calibrating relevance scores between a query request vector embedding and candidate clinical guideline vector embeddings. Similarity score calculator 144 calculates a similarity between vector embeddings for a query request and candidate clinical guidelines. To measure similarity, a distance measure or similarity metric is chosen. Common distance measures include Euclidean distance, cosine similarity, and Jaccard similarity. When dealing with large data sets, an index may be created to speed up the search process. An index is a data structure that organizes the data in a way that allows for efficient retrieval of similar items.

In one or more embodiments, prior authorization decision support engine 104 includes a questionnaire prefill module 146. Questionnaire prefill module 146 refers to hardware and/or software configured to perform operations described herein for automatically populating questionnaires 126 with patient-specific data derived from patient data 110. Questionnaire prefill module 146 enables automated alignment between extracted patient evidence and the structured question fields generated by the questionnaire generator module 136, thereby reducing manual clinician input and improving completeness of medical necessity evaluations.

In one or more embodiments, questionnaire prefill module 146 accesses parsed and chunked clinical data from EHR and external sources, including demographics, encounter summaries, diagnoses, procedures, laboratory results, imaging findings, medications, and clinical notes. Each data element may be preprocessed into a normalized representation and encoded into a patient data vector embedding. Questionnaire prefill module 146 may access clinical guideline rules 118 to identify mappings between rule criteria and questionnaire items, ensuring that each question is evaluated against the appropriate evidence domain.

In one or more embodiments, questionnaire prefill module 146 computes similarity scores 122 between question embeddings and patient data embeddings to determine if specific subsets of patient data are relevant to answering each question. The module may apply threshold scores to classify similarity values as affirmative (“Yes”), negative (“No”), or indeterminate (“Insufficient Information”). For example, a question, such as, “Has the patient completed conservative medical management?” may be automatically prefilled with a “Yes” response if the similarity score between relevant patient medication records and the conservative management guideline exceeds a defined threshold.

In one or more embodiments, questionnaire prefill module 146 generates structured explanations and evidence rationales for each prefilled response. These justifications may include the underlying data excerpts, source references (e.g., encounter date, provider note ID, lab test name), and/or similarity or confidence scores. Prefilled responses and corresponding explanations may be formatted as structured objects (e.g., FHIR QuestionnaireResponse resources) with traceable provenance attributes to support payer review and audit transparency. Questionnaire prefill module 146 may include adaptive validation logic that detects incomplete or conflicting evidence across data sources. When similarity scores for multiple candidate data subsets exceed a threshold or conflict, the module may invoke a GenAI model to reconcile ambiguity through contextual reasoning or evidence summarization. The resulting refined prefill may then be submitted for deterministic validation by the prior authorization decision support engine 104.

In one or more embodiments, questionnaire prefill module 146 employs feedback integration through a feedback mechanism, whereby reviewer corrections or payer adjudication results are used to recalibrate similarity thresholds, retrain embedding models, or adjust the mapping between patient data elements and guideline criteria. Over time, the module improves prefill accuracy, contextual relevance, and confidence calibration through continuous learning.

In one or more embodiments, outputs of questionnaire prefill module 146 are persisted as versioned records within a clinical data repository, each linked to its originating questionnaire, clinical guideline rule, and/or GenAI model configuration. Prefilled questionnaires may be directly consumed by an evaluation module 148 for automated determination of medical necessity and subsequent generation of a prior authorization request 130.

In one or more embodiments, prior authorization decision support engine 104 includes evaluation module 148. Evaluation module 148 refers to hardware and/or software configured to perform operations described herein for generating evaluative determinations and predictive inferences regarding the medical necessity and clinical appropriateness of proposed medical procedures 114 for patient conditions 112. Evaluation module 148 may integrate outputs from questionnaire prefill module 146 and/or clinical guideline rules 118 to produce structured evaluations 128 representing decision outcomes.

In one or more embodiments, evaluation module 148 receives a completed or partially prefilled clinical guideline questionnaire and applies one or more decision models to determine if the collected evidence satisfies all required medical necessity criteria defined in the corresponding clinical guideline rules 118. Decision models may include deterministic rule engines, statistical classifiers, or GenAI models 156 trained on historical authorization outcomes, guideline text, and annotated medical cases. Evaluation module 148 may compute one or more evaluation scores or confidence metrics that quantify the degree of compliance between patient-specific data and the evidence-based guideline criteria. For example, evaluation module 148 may generate a probabilistic output (e.g., 0.91 likelihood of meeting medical necessity) or a categorical label (e.g., “Meets,” “Does Not Meet,” or “Requires Review”). Evaluation module 148 may compute intermediate scores per question or criterion, aggregating the scores hierarchically to derive an overall authorization prediction.

In one or more embodiments, evaluation module 148 applies threshold scores to the computed evaluation metrics to produce discrete outcomes. If the aggregate score exceeds a predefined threshold, evaluation module 148 may automatically classify the request as meeting medical necessity; otherwise, evaluation module 148 may mark the case for human review. The thresholds may be configurable by payer, procedure type, or guideline authority and may be adaptively tuned through a feedback mechanism.

In one or more embodiments, evaluation module 148 includes an explanation subsystem configured to generate natural-language justifications for each evaluation result. Explanations may identify the supporting evidence, associated question responses, and applicable guideline citations. For example, if a denial prediction is issued, evaluation module 148 may produce a rationale, such as, “Criterion A not satisfied—patient age (1 year) below required minimum (2 years).” Each explanation may reference provenance metadata, including patient data source, encounter date, and document identifier, to support payer audit and traceability.

In one or more embodiments, evaluation module 148 operates in a hybrid inference mode, wherein deterministic rule-based evaluation and GenAI reasoning operate in tandem. The deterministic engine may enforce explicit guideline conditions, and the GenAI reasoning component may provide contextual interpretation for ambiguous or narrative inputs (e.g., interpreting free-text provider notes that imply prior treatment failure). The combined outputs may be reconciled through weighted voting or confidence aggregation functions to ensure both accuracy and explainability.

In one or more embodiments, evaluation module 148 produces evaluation artifacts in standardized, interoperable formats (e.g., HL7 FHIR Task, CoverageEligibilityResponse, or custom JSON structures). Each evaluation artifact may include the outcome classification, confidence scores, explanation text, and associated metadata. Evaluation module 148 may transmit these artifacts to a prior authorization request generator or directly to payer adjudication interfaces for automated processing.

In one or more embodiments, evaluation module 148 employs a feedback-driven refinement loop, in which post-adjudication feedback, reviewer overrides, and appeal outcomes are analyzed to improve model accuracy and threshold calibration. This continuous learning cycle enables adaptive optimization of evaluation models, enhancing precision, reducing false determinations, and aligning predictions with evolving payer and clinical standards.

In one or more embodiments, prior authorization decision support engine 104 includes a reranking mechanism 150. Reranking mechanism 150 refers to hardware and/or software configured to perform the operations described herein for reordering a candidate subset of patient data. Reranking mechanism 150 may assist in maximizing decision-time relevance and policy compliance in the prior-authorization workflows. Reranking mechanism 150 may ingest a list of candidates (e.g., guideline fragments, plan rules, patient-evidence pointers) accompanied by calculator outputs (namely a unified similarity score in [0,1]), per-feature contributions (dense/lexical/code overlaps, temporal/unit proximity), and candidate metadata (issuer, policy identifier/version/effective dates, product line and site-of-care applicability, clause type, section/paragraph anchors, chunk lineage, and provenance hashes).

In one or more embodiments, reranking mechanism 150 applies deterministic filters that zero or exclude items failing hard constraints (outside effective/sunset windows, product-line/site mismatch, issuer precedence violations). Reranking mechanism 150 may collapse near-duplicates using shingling/content hashes and version lineage (retaining the highest-utility representative). Reranking mechanism 150 may compose a rerank feature vector per candidate that includes the following: (i) the calculator’s calibrated similarity score; (ii) policy-fit indicators (effective-date gap, product-line/site compatibility); (iii) clinical specificity features (presence of thresholds, temporal windows, step-therapy or contraindication gates); (iv) structural features (heading depth, table vs. narrative, code-table coverage); (v) recency/version features; and/or (vi) diversity/conflict signals (coverage vs non-coverage pairings for the same code/indication). A learning-to-rank model (pointwise/pairwise/listwise) and/or a cross-encoder scorer (joint query-candidate transformer) may produce a refined relevance score. Reranking mechanism 150 may apply score fusion to combine a refined score with a calculator’s score via learned or calibrated weights to yield a final confidence.

In one or more embodiments, reranking mechanism 150 enforces list-level constraints and diversification (e.g., Maximal Marginal Relevance). This may ensure that a top-K set covers complementary rationale types (coverage indications, documentation requirements, coding tables) and avoids redundant fragments from the same clause/version. Tie-breakers ensure determinism (priority: higher fused score → closer effective date → issuer precedence → section priority → stable chunk ID). Outputs include the following: a ranked bundle with per-item final scores and confidence intervals; explanations listing the dominant features that changed their order relative to the similarity score calculator; resolved conflict sets (e.g., overlapping coverage/non-coverage clauses) with the applied precedence rationale; and anchors (page/section/offsets) suitable for citation in downstream rationales. Reranking mechanism 150 supports batch and streaming modes, batched cross-encoding, early-exit heuristics, and/or caching for frequent code/indication queries.

In one or more embodiments, reranking mechanism 150 employs rerank-english-v3.0 as the reranking model.

In one or more embodiments, prior authorization decision support engine 104 includes a selection mechanism 152. Selection mechanism 152 refers to hardware and/or software configured to perform the operations described herein for selecting relevant subsets, or chunks, of patient data from a set of patient data for answering particular questions of a clinical guideline questionnaire. Selection mechanism 152 may select, as relevant, a subset of patient data associated with a patient data vector embedding having similarity score with a question vector embedding above a threshold score. Alternatively, selection mechanism 152 may select, as relevant, a top number of subsets of patient data associated with patient data vector embeddings ranked by similarity score with the question vector embedding.

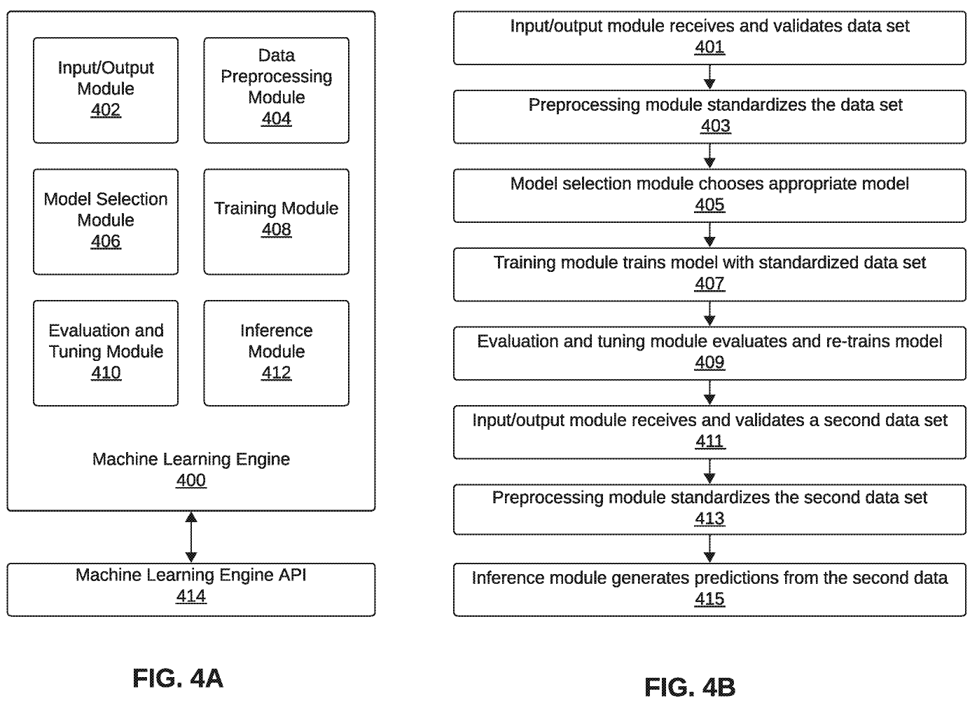

In one or more embodiments, prior authorization decision support engine 104 includes machine learning (ML) engine 154. ML engine 154 refers to hardware and/or software configured to perform the operations described herein for training and/or applying ML models. The structure and function of ML engines are described in detail below in Section 7, titled MACHINE LEARNING ARCHITECTURE and Section 8, titled MACHINE LEARNING ENGINE OPERATIONS, that reference FIGS. 4A and 4B, respectively.

In one or more embodiments, prior authorization decision support engine 104 includes GenAI models 156. GenAI models 156 refer to hardware and/or software configured to perform the operations described herein for determining a prior authorization decision for medical procedures for patients. GenAI models 156 may be an LLM, e.g., command-r-plus-08-2024 (a refined LLM for reasoning tasks) and/or command-xlarge-nightly (a cutting edge LLM trained nightly for the latest updates and enhanced performance). GenAI models are described in detail below in Section 9, titled GENERATIVE AI MODELS.

In one or more embodiments, interface 106 refers to hardware and/or software configured to facilitate communications between a user and prior authorization decision support engine 104. Interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of interface 106 are specified in different languages. The behavior of user interface elements is specified in a dynamic programming language such as JavaScript. The content of user interface elements is specified in a markup language, such as hypertext markup language (HTML) or XML User Interface Language (XUL). The layout of user interface elements is specified in a style sheet language such as Cascading Style Sheets (CSS). Alternatively, interface 106 is specified in one or more other languages, such as Java, C, or C++.

In one or more embodiments, third-party payer 108 refers to a computing system, entity, or external service responsible for receiving, adjudicating, and responding to prior authorization requests 130 generated by the prior authorization decision support engine 104. Third-party payer 108 may represent a health insurance provider, managed care organization, government payer (e.g., Medicare, Medicaid), or self-funded employer plan that administers reimbursement decisions for covered medical procedures 114. Third-party payer 108 may include interfaces and APIs for receiving structured authorization submissions. Such interfaces may adhere to interoperability standards, including X12 278 Health Care Services Review, HL7 FHIR PriorAuthorization, or proprietary web service specifications. Incoming requests may include structured data elements, such as patient demographics, clinical evidence summaries, questionnaire responses, evaluation results, CPT/HCPCS and ICD-10 codes, and supporting documentation references.

In one or more embodiments, third-party payer 108 includes an adjudication engine configured to evaluate authorization requests based on applicable clinical guideline rules 118, payer coverage policies, and utilization management criteria. The adjudication engine may perform rule-based, statistical, or AI-assisted determinations to classify requests as “approved,” “denied,” “pended,” or “requires manual review.” Each adjudication decision may be accompanied by one or more rationale statements referencing specific guideline conditions or missing documentation criteria.

In one or more embodiments, third-party payer 108 communicates feedback to the prior authorization decision support engine 104 in response to submitted requests. The feedback may include approval or denial codes, adjudication rationales, and audit references. The feedback may be stored as structured data for continuous learning by questionnaire prefill module 146 and/or evaluation module 148, enabling adaptive refinement of model thresholds, similarity scores, and prompt configurations. Metadata associated with third-party payer 108 may include payer identification (e.g., NAIC ID, payer code), plan type (commercial, Medicare Advantage, Medicaid), network coverage region, policy versions, and effective dates. Each payer record may also maintain links to associated clinical guideline repositories, rule libraries, and authorization history logs for audit and compliance tracking.

In one or more embodiments, third-party payer 108 provides secure, bidirectional communication channels for status updates, additional documentation requests, or secondary review notifications. Authentication and data exchange may employ industry-standard security protocols (OAuth 2.0, TLS, or FHIR SMART authorization) to ensure confidentiality and integrity of protected health information (PHI). Third-party payer 108 may integrate with external analytics modules to track decision turnaround time, policy utilization trends, and denial pattern analytics. Aggregated feedback and outcome data may be shared with providers or regulatory bodies to promote transparency and improve policy accuracy.

### 3. Leveraging Genai to Extract and Parse Structured and Unstructured data for use by Genai to Automatically Generate Evaluations of Medical necessity for Medical Procedures for Patient Conditions

FIG. 2A illustrates an example set of operations for leveraging GenAI to extract and parse structured and unstructured data for use by GenAI to automatically generate evaluations of medical necessity for medical procedures for patient conditions in accordance with one or more embodiments. One or more operations illustrated in FIG. 2A may be modified, rearranged, or omitted. Accordingly, the particular sequence of operations illustrated in FIG. 2A should not be construed as limiting the scope of one or more embodiments.

One or more embodiments access patient data of a patient, the patient data identifying a patient condition (Operation 202). Accessing patient data may include identifying the target patient, authenticating the requesting module or user, constructing and executing a federated query, and/or retrieving results in normalized formats (e.g., FHIR JSON, XML, or HL7 messages). The system may perform patient identity resolution using one or more unique identifiers, such as MRN, payer subscriber ID, national provider identifier (NPI), or combinations of demographics (e.g., name, date of birth, gender).

In one or more embodiments, access operations are secured using industry-standard authentication and authorization protocols, including Open Authorization 2.0 (OAuth2), SMART-on-FHIR, or signed API keys. An authorization layer ensures that the retrieval of patient data is restricted to authenticated and authorized entities, such as registered healthcare organizations or payer systems. The system may log each access event, capturing requester credentials, timestamp, and data scope for compliance with HIPAA and audit traceability requirements. Once authenticated, the system may issue queries to one or more data sources through FHIR endpoints (e.g., Condition, Observation, Procedure, MedicationStatement, Encounter, DocumentReference resources) or via proprietary EHR APIs. Returned data may include structured JSON payloads, semi-structured documents, or unstructured text (e.g., clinical notes). These responses may be preprocessed to normalize formats, extract semantic content, and/or segment patient information into machine-interpretable units for downstream processing.

One or more embodiments access (i) a clinical guideline questionnaire and (ii) a set of clinical guideline rules, each associated with a clinical guideline for a medical procedure associated with the patient condition (Operation 204). As will be described in detail with reference to FIG. 2B, the system may use ML, e.g., GenAI models, to generate clinical guideline questionnaires associated with clinical guidelines. Clinical guideline rules for each clinical guideline are provided with the respective clinical guidelines.

One or more embodiments populate the clinical guideline questionnaire with a set of patient data to generate a prefilled questionnaire (Operation 206). As will be described in detail with reference to FIG. 2C, the system may use ML to determine patient data most relevant to answering questions of the clinical guideline questionnaire. The system populates the questions of the clinical guideline questionnaire with the relevant patient data.

One or more embodiments apply a first ML model to the prefilled questionnaire and the set of clinical guideline rules to generate an evaluation that includes a determination of medical necessity for the medical procedure for the patient (Operation 208). The first ML model may comprise a GenAI model, implemented as an LLM or hybrid inference engine optimized for clinical reasoning tasks. Example LLMs may include command-r-plus-08-2024 (a refined LLM for reasoning-intensive operations), command-xlarge-nightly (a cutting-edge LLM continuously trained for model updates and enhanced accuracy), and OpenAI models, such as GPT-4o and GPT-o1 (multi-modal reasoning architectures capable of contextual understanding across structured and unstructured medical data).

In one or more embodiments, the system trains or fine-tunes the first ML model to specialize in prior authorization evaluations. The system may perform fine-tuning using curated datasets comprising historical prior authorization decisions, payer medical policies, and annotated clinical guideline documents. Training data may include examples mapping patient-specific evidence (e.g., diagnosis, test results, treatment history) to corresponding guideline conditions and adjudication outcomes (e.g., approved, denied, pending). The fine-tuned model thus learns to recognize both explicit and implied satisfaction of guideline criteria, including handling of ambiguous or partially satisfied conditions.

In one or more embodiments, preparing the first ML model for inference includes constructing a structured prompt that encodes (i) the text and metadata from the prefilled questionnaire, (ii) the logical structure and conditions from the clinical guideline rules, and/or (iii) relevant patient evidence extracted from patient data. The prompt may be formatted as a structured input template or a multimodal token sequence, including question-answer pairs, condition descriptors, coded terminologies, and threshold criteria. The prompt serves as a contextual instruction set for the model, guiding it to generate a grounded and guideline-compliant determination.

In one or more embodiments, the system applies the first ML model to this composite input to perform reasoning over both structured (e.g., Boolean or numeric questionnaire responses) and unstructured (e.g., narrative justifications, provider notes) data. The model evaluates the alignment between the patient-specific responses and the guideline-based conditions using internal attention mechanisms and learned representations of clinical semantics. The output of the model is an evaluation artifact that includes a predicted determination (e.g., “Meets Medical Necessity,” “Does Not Meet,” or “Requires Review”) along with a structured justification or explanation for the outcome.

In one or more embodiments, the first ML model may incorporate RAG to minimize hallucinations and improve factual grounding. During inference, the model retrieves relevant text passages, data excerpts, and/or coded entities from a clinical guideline repository and a patient data repository. The system may embed these retrieved elements into the model’s context window, ensuring the generated evaluation references authentic guideline language and verified patient evidence rather than inferred approximations.

In one or more embodiments, the first ML model employs hybrid reasoning layers that combine generative inference with deterministic rule validation. The deterministic layer may enforce threshold checks (e.g., numeric limits, age, or duration criteria) defined in the clinical guideline rules, while the generative reasoning layer may interpret narrative context (e.g., “patient reports persistent symptoms despite conservative treatment”) to determine functional equivalence to guideline conditions. This hybrid evaluation process enables the system to address both objective and contextual evidence with precision.

In one or more embodiments, the output evaluation includes multiple components: (i) a prediction of whether the medical procedure satisfies medical necessity criteria; (ii) a clinical indication summary highlighting the key evidence influencing the decision; (iii) explanatory reasoning detailing which criteria were met or unmet; and/or (iv) metadata fields, including model version, prompt reference, confidence score, and evidence provenance. The system may encode these artifacts in structured formats (e.g., JSON, XML, HL7 FHIR Task) suitable for downstream processing by third-party payer systems.

In one or more embodiments, the generated evaluation is subsequently validated by a feedback mechanism that compares the GenAI output against deterministic rule-engine outcomes or historical payer responses to ensure internal consistency and compliance. Discrepancies may trigger threshold recalibration, additional retrieval passes, and/or flagging for human reviewer verification. The system may then persist the validated evaluation in a prior authorization decision log and use the validated evaluation to generate a corresponding prior authorization request.

By combining generative reasoning, retrieval augmentation, rule-based validation, and structured prompt orchestration, the system achieves a higher degree of accuracy, transparency, and explainability than conventional rule-based or manual review processes. This integrated approach enables context-aware, evidence-grounded medical necessity determinations across heterogeneous data sources, improving both the speed and quality of prior authorization workflows.

One or more embodiments generate an evaluation that includes a determination of medical necessity for the medical procedure associated with the patient condition (Operation 210). An evaluation module may invoke a first ML model, i.e., GenAI model or hybrid inference engine, to process a structured prompt that includes the prefilled questionnaire, the clinical guideline rules, and/or the relevant patient data.

In one or more embodiments, the first ML model employs a multi-input reasoning architecture configured to jointly attend over the structured questionnaire fields and rule-based text segments. The prompt may include the patient-specific evidence (answers, supporting explanations, extracted clinical findings) and the encoded guideline criteria (conditions, thresholds, exceptions). In response, the model generates a structured evaluation artifact that predicts if the medical procedure is medically necessary given the patient’s clinical context.

In one or more embodiments, the evaluation includes (i) a binary or categorical classification (e.g., “Meets Medical Necessity,” “Does Not Meet,” or “Requires Review”); (ii) a confidence or probability score quantifying the likelihood that the procedure satisfies the guideline criteria; and/or (iii) an explanation or reasoning sequence that outlines the evidence-based justification for the prediction. The explanation may include a clinical indication summary that contextualizes the decision outcome by describing the relevant aspects of the patient’s condition, diagnostic findings, and/or treatment history. For example, an evaluation for a “Transanal Irrigation” procedure may output the following reasoning: “Criterion A not met—patient is 1 year old; guideline requires minimum age of 2 years. Other conditions (neurogenic bowel dysfunction, chronic constipation) are satisfied.”

In one or more embodiments, to improve explainability and accuracy, the first machine learning model implements a RAG framework. The RAG layer may dynamically retrieve text segments or structured data snippets from clinical guideline and/or patient data repositories during inference. Retrieved content may include actual excerpts (e.g., guideline clauses, laboratory reports, or problem list entries) that are surfaced in the output to substantiate the reasoning. This retrieval process grounds the model’s output in verifiable evidence, thereby minimizing hallucinations and ensuring that each generated statement corresponds to documented facts.

In one or more embodiments, the evaluation generation process includes evidence alignment scoring, in which the system computes similarity scores between the patient’s evidence embeddings and the corresponding guideline embeddings to validate the GenAI model’s outputs. The similarity results are incorporated into the evaluation metadata to provide both quantitative and narrative justifications for the decision outcome. This hybrid approach, i.e., combining deterministic evidence scoring with generative reasoning, produces evaluations that are both machine-verifiable and human-auditable.

In one or more embodiments, the generated evaluation is structured as a machine-readable artifact (e.g., HL7 FHIR Task, CoverageEligibilityResponse, or JSON schema) that includes numerous fields, such as procedure_code, decision_outcome, confidence_score, explanation, referenced_guideline, supporting_evidence, and timestamp. The system may store the evaluation in a local or distributed data repository.

In one or more embodiments, the evaluation model may employ fine-tuned parameters optimized for healthcare-specific natural language inference (NLI), incorporating domain embeddings trained on guideline corpora, payer policies, and/or annotated prior authorization cases. The model may leverage transformer-based architectures (e.g., encoder-decoder or mixture-of-experts models) trained to generate structured outputs constrained by schema validation layers to prevent syntactic or semantic errors.

In one or more embodiments, post-generation, the evaluation undergoes a validation step by a feedback mechanism. The feedback mechanism may compare the GenAI output against deterministic rule-engine results for consistency. Discrepancies between the generative reasoning and the rule-based evaluation may trigger a reconciliation operation that either adjusts the confidence weighting or flags the record for human review. This cross-verification ensures robust quality control while maintaining system transparency.

One or more embodiments determine if the patient condition satisfies the medical necessity for the medical procedure (Operation 212). The system provides the determination of whether a medical procedure is medically necessary or not in the evaluation. The system may provide the determination through text or other visual display, e.g., a checkmark or green field for medically necessary or an “X” or red field for not medically necessary.

When the system determines that the patient condition does not satisfy the medical necessity for the medical procedure, one or more embodiments update the patient data (Operation 214). Updating the patient data may involve initiating or recommending actions to collect additional clinical evidence, perform follow-up assessments, or modify data representations to reflect ongoing patient care.

In one or more embodiments, an evaluation module generates an evaluative outcome indicating that one or more guideline criteria remain unsatisfied. The system may invoke a feedback mechanism or an update controller to identify which specific data elements contributed to the unmet condition. The feedback may include structured metadata identifying missing or insufficient attributes, such as an absent laboratory result, an age-based eligibility gap, or incomplete documentation of conservative therapy.

In one or more embodiments, updating patient data includes initiating clinical or administrative follow-up workflows. For example, the system may determine that the patient’s condition fails to meet an age or duration requirement specified in the clinical guideline rules. In such instances, the system may record an expected re-evaluation date, corresponding to when the patient will reach the minimum age or when sufficient treatment duration has elapsed (e.g., “90 days after initiation of physical therapy”). The system may schedule an automated reminder or flag within the EHR to reprocess the authorization request upon data maturity.

In another example, when laboratory or diagnostic data required for medical necessity verification is unavailable or outdated, the system may generate an order recommendation or task request to prompt the ordering provider to perform the missing test. The recommendation may include the test name, relevant LOINC code, and guideline reference that justifies the data requirement. For instance, if a guideline requires a hemoglobin A1c measurement within the last 90 days, the system may generate a task, such as, “Order HbA1c test—required for procedure authorization under CG-MED-95.”

In one or more embodiments, updating patient data involves incorporating new results, clinical notes, or third-party data into the patient data. The system may continuously monitor connected data sources (EHR, FHIR endpoints, HIE feeds, or wearable devices) and trigger re-ingestion and re-chunking of updated records. The system may reprocess the incoming data, normalize formats, and refresh associated vector embeddings. The system may then re-evaluate these embeddings to determine if the updated evidence now satisfies the guideline thresholds.

In one or more embodiments, updating patient data includes adjusting the confidence weightings and similarity scores associated with previously incomplete or ambiguous evidence. For example, a previously low similarity score for “failed conservative management” may increase after ingestion of new provider notes describing additional therapy sessions. The system may dynamically recompute these similarity metrics and re-score the medical necessity evaluation, potentially transitioning the outcome from “Does Not Meet Criteria” to “Meets Criteria.”

In some embodiments, a feedback mechanism captures payer adjudication feedback, reviewer notes, or appeal outcomes that indicate why a prior authorization request was denied or delayed. This information may trigger rule-based or AI-driven updates to patient data mappings, prompting targeted data acquisition or record augmentation to satisfy future submissions. Feedback may also update internal knowledge graphs linking conditions, procedures, and guideline criteria, thereby improving future decision accuracy.

If the patient condition satisfies medical necessity for the medical procedure, then one or more embodiments generate a prior authorization request for the medical procedure for the patient (Operation 216). The system may automatically construct a structured prior authorization request suitable for electronic submission to a third-party payer. The system may compile all required elements of the authorization request, including the following: (i) patient demographic details (name, date of birth, sex, identifiers, insurance information); (ii) clinical presentation summarizing the patient’s symptoms, findings, and relevant encounters; (iii) procedure justification, referencing the evaluation’s rationale and satisfied guideline criteria; (iv) medical history and treatment chronology; (v) diagnosis description, encoded using standardized terminologies (ICD-10-CM, SNOMED CT); (vi) post-operative or follow-up plan; and (vii) billing and procedural identifiers, such as CPT/HCPCS codes, ICD-10-PCS codes, and payer plan identifiers. Each section of the authorization request may correspond to structured fields defined by payer interface specifications (e.g., HL7 FHIR PriorAuthorization, X12 278 transaction).

In one or more embodiments, a questionnaire prefill module and/or evaluation module provide structured data outputs that populate the authorization request automatically. The system may extract verified patient attributes, procedure codes, and justification text directly from patient data and/or the evaluation. The system may embed evidence passages retrieved through RAG and verified similarity scores into the justification narrative, ensuring that each request cites the precise data supporting medical necessity (e.g., “HbA1c 7.9% measured 05/12/2025—meets glycemic control threshold for diabetic foot ulcer treatment”).

In one or more embodiments, a prior authorization request generator module validates the assembled content through schema and semantic checks. Schema validation ensures that all required payer fields are present and syntactically correct, while semantic validation verifies consistency between coded data and narrative reasoning (e.g., the diagnosis code aligns with the described condition, and the procedure code corresponds to the justified intervention). The system may enrich the request with provenance metadata, including generation timestamp, model version, source document identifiers, and digital signatures, to support audit traceability and compliance with payer and regulatory requirements (e.g., HIPAA, CMS).

In one or more embodiments, the system formats the completed request for submission via standardized electronic interfaces. For example, the request may be serialized as a FHIR PriorAuthorization resource, an X12 278 message, or a secure JSON payload transmitted through payer APIs. Authentication and transmission may employ OAuth 2.0 tokens, TLS encryption, and SMART-on-FHIR authorization. Upon successful submission, the system may receive an acknowledgment or transaction ID from a third-party payer, which is stored in association with the originating evaluation for end-to-end traceability.

In one or more embodiments, the system generates both machine-readable and human-readable representations of the authorization request. The machine-readable representation enables direct ingestion by payer systems, while the human-readable version, rendered as a formatted document or dashboard view, summarizes the key elements of the request, including decision rationale, evidence excerpts, and clinical guideline citations. This dual representation improves transparency for providers and facilitates reviewer validation.

In one or more embodiments, the system incorporates an adaptive submission controller that selects the optimal payer interface based on plan ID, network, and supported transaction standards. The controller may automatically route follow-up messages, such as requests for additional documentation or notification of decision updates, through bidirectional communication channels defined by the third-party payer.

One or more embodiments submit the prior authorization request for the medical procedure for the patient to a third-party payer (Operation 218). Once the prior authorization request has been generated and validated, the system transmits the request to the corresponding payer endpoint for adjudication. A prior authorization submission module may automatically determine an optimal submission channel based on payer metadata (e.g., payer ID, network, and supported interoperability standards). Transmission protocols may include electronic data interchange (EDI) using X12 278 transaction sets, FHIR-based RESTful APIs (e.g., PriorAuthorization and ClaimResponse resources), or secure web portal automation using authenticated session workflows.

In one or more embodiments, the prior authorization request is serialized into a structured, machine-readable format. The submission payload may include patient identifiers, provider credentials, procedure and diagnosis codes (CPT/HCPCS and ICD-10-CM), medical necessity justification text, clinical evidence summaries, supporting attachments (e.g., PDFs, diagnostic results, imaging), and metadata fields, such as timestamps, digital signatures, and model version identifiers. The system may encrypt the payload and apply authentication tokens (e.g., OAuth 2.0 bearer tokens, JWT assertions, or mutual-TLS certificates) to ensure secure transmission of protected health information (PHI) in compliance with HIPAA and payer security requirements.

Upon successful transmission, the third-party payer may generate a synchronous or asynchronous acknowledgment message. The acknowledgment may include an authorization control number, payer transaction ID, and a preliminary status indicator (e.g., “accepted,” “pending review,” “denied,” or “requires additional documentation”). The system may parse and store the acknowledgment response in association with the originating prior authorization request and the supporting evaluation to maintain a continuous audit trail.

In one or more embodiments, the system maintains bidirectional communication channels with the payer to track adjudication progress. The system may retrieve status updates via polling mechanisms, where the system periodically queries the payer’s API for status changes, or via subscription-based event notifications, where the system registers a callback endpoint to receive real-time updates when the payer modifies the authorization status. Updates may include changes to adjudication state, reviewer comments, or requests for additional documentation.

In one or more embodiments, when a response indicates that the authorization is pending or requires additional documentation, the system may automatically trigger a feedback cycle through a feedback mechanism. The feedback may identify missing evidence, required clarification, or unverified data elements. The system may then invoke a questionnaire prefill module and/or parsing mechanism to collect or re-extract relevant patient data, perform additional retrieval-augmented queries, and regenerate the authorization request with the new information.

In one or more embodiments, when the authorization request is approved, the system may record the payer-assigned authorization number, effective date, and/or coverage period within patient data and the provider’s EHR. The system may persist this data in structured form (e.g., FHIR Coverage or ClaimResponse resources) to enable downstream billing, scheduling, and claims submission workflows. If the request is denied, the system may log the denial reason codes, generate an explanation summary, and optionally initiate an appeal workflow or re-submission procedure after updating relevant patient data.

In one or more embodiments, the system includes a monitoring and notification subsystem configured to alert the submitting provider or care team in real time upon status changes. Notifications may include authorization numbers, decision summaries, pending document requests, or denial explanations. Alerts may be delivered via secure messaging, dashboard updates, or integrated EHR notifications, ensuring transparency and operational efficiency in care coordination.

One or more embodiments, in response to receiving approval from the third-party payer, treat the patient in accordance with the medical procedure (Operation 220). Upon receipt of the authorization response, the system may parse the adjudication message, such as an HL7 FHIR ClaimResponse resource or an X12 278 acknowledgment, and extract the decision status, approval code, authorization control number, effective period, and/or payer comments. The system may then update the patient’s EHR to reflect the payer’s authorization outcome (e.g., approved, denied, or pending). The EHR update may include timestamped metadata identifying the payer source, response transaction ID, decision rationale, and linkage to the supporting evaluation for audit traceability.

In one or more embodiments, when the system determines the prior authorization request for the medical procedure is approved, the system may automatically initiate or facilitate preparation for the medical procedure. A procedure preparation module may trigger downstream workflows based on predefined care-path templates or provider-configured automation rules. Initiation of preparations may occur either automatically, i.e., through integration with scheduling, resource management, and/or inventory systems, or may require manual confirmation by authorized medical personnel via an interactive interface element within the prior authorization request interface. The interface may display the approval details, procedure summary, and available scheduling slots, allowing clinicians to confirm readiness or modify timing.

In one or more embodiments, initiating preparation for the medical procedure may include: (i) scheduling the procedure, (ii) updating the treatment plan, and/or (iii) notifying relevant parties. Scheduling the procedure may include reserving an operating room or procedural suite, allocating anesthesia and surgical personnel, coordinating diagnostic or pre-operative testing, and confirming pre- and post-operative accommodations. The scheduling component may interface with a facility’s EHR scheduling API or a standalone clinical resource planning system. Updating the treatment plan may involve amending the patient’s care plan record in the EHR to initiate, modify, or discontinue medications, therapies, or supportive interventions in anticipation of the procedure. For example, the system may automatically issue medication reconciliation prompts (e.g., “Discontinue anticoagulant 48 hours before surgery.”) based on procedural protocols or clinical guideline rules. Notifying relevant parties may include primary providers, specialists, nursing teams, and billing staff. Notifications may be delivered through secure messaging systems, EHR task lists, or external communication platforms, each including the authorization number, procedure date, and pre-authorization reference details.

In one or more embodiments, the system propagates the approval to ancillary modules, including inventory management systems, to pre-order required devices or supplies. In addition, the system propagates billing systems to initialize claims pre-authorization records and propagates patient engagement portals to display approval status and pre-procedure instructions. The system may also monitor payer validity windows (e.g., authorization valid for 30 days) and automatically alert staff if the procedure is not completed within the approved timeframe.

In one or more embodiments, when the system receives a denial or pending response, the system updates the EHR accordingly and may trigger an automated follow-up workflow through a feedback mechanism. This may include generating a denial summary, highlighting the specific unmet criteria, and prompting the provider to supply additional documentation or initiate an appeal. If the denial reason corresponds to missing or outdated data (e.g., expired lab results), the system may refresh patient data and regenerate a revised authorization request.

### 4. Leveraging Genai to Generate Questionnaires for use by Genai in Evaluating Medical necessity of Medical Procedures for Patient conditions

FIG. 2B illustrates an example set of operations for leveraging GenAI to generate questionnaires for evaluating medical necessity of medical procedures for patient conditions in accordance with one or more embodiments. One or more operations illustrated in FIG. 2B may be modified, rearranged, or omitted. Accordingly, the particular sequence of operations illustrated in FIG. 2B should not be construed as limiting the scope of one or more embodiments.

One or more embodiments access the clinical guideline from a plurality of clinical guidelines for the medical procedure (Operation 204a). Clinical guidelines are included in resources provided at healthcare provider websites and are available in PDF format. The system may use web scraping to download the clinical guidelines from the website. BeautifulSoup, or other similar tools, may be used to parse and navigate an HTML structure of the website. The system may use Selenium, or other similar tools, to handle dynamic content and automate interactions for retrieving required files.

One or more embodiments prompt a second ML model to generate the clinical guideline questionnaire for the clinical guideline for the medical procedure (Operation 204b). The second ML model may be a generative model, e.g. LLM. The second ML model may be the same or different from the first ML model used to determine medical necessity. The system may train or fine-tune the second ML model to generate a clinical guideline questionnaire for the clinical guideline associated with the medical procedure. The system may further prompt the second ML model by providing a prompt to the second ML model that includes a desired format for the questionnaire.

One or more embodiments apply the second ML model to the clinical guideline for generating the clinical guideline questionnaire (Operation 204c). The second ML model may comprise a GenAI model, such as an LLM, fine-tuned for knowledge extraction, rule structuring, and question synthesis from unstructured or semi-structured clinical text.

In one or more embodiments, the system applies the second ML model to the clinical guideline text, which may be represented as a parsed and chunked corpus. The parsed guideline text may include numerous sections, such as “When Covered,” “When Not Covered,” “Clinical Indications,” “Contraindications,” “Documentation Requirements,” and “Coding Information.” The second ML model may process this content to extract logical rule structures and conditional dependencies representing medical necessity criteria, clinical prerequisites, contraindications, and documentation requirements.

One or more embodiments generate, the clinical guideline, (a) the clinical guideline questionnaire, and (b) the set of clinical guideline rules (Operation 204d). The second ML model may transform the extracted content into structured question templates that collectively form a clinical guideline questionnaire. Each question template may encode the logical condition of a rule as a machine- and human-interpretable question, associated with a specific evidence domain (e.g., diagnosis, test result, treatment history, patient age, or contraindication). For example, the guideline statement, “Procedure X is medically necessary when conservative management for ≥6 weeks has failed.” may be transformed into a question, such as “Has the patient completed at least 6 weeks of conservative medical management without adequate improvement?”

In one or more embodiments, the model generates metadata for each question, including (i) question identifiers and hierarchy levels (e.g., parent/child or dependent/branching relationships), (ii) logical operators (AND, OR, NOT) representing guideline dependencies, (iii) value types and permissible responses (Boolean, numeric, categorical, date/time), and/or (iv) code bindings linking each question to standardized terminologies, such as ICD-10-CM, SNOMED CT, LOINC, CPT/HCPCS, and RxNorm. Each generated question may include a source citation (e.g., guideline ID, section title, and paragraph reference) and a provenance record capturing model version, prompt configuration, and retrieval sources, ensuring traceability and compliance with payer audit standards.

In one or more embodiments, the system employs RAG during questionnaire synthesis to improve accuracy and reduce hallucination. The RAG subsystem retrieves relevant text passages and rule fragments from a clinical guideline repository or clinical guideline rules and provides the rule fragments as factual context to the second ML model. The model may generate each question with direct references to the retrieved text, ensuring the resulting questionnaire remains semantically consistent with the original guideline.

In one or more embodiments, the second ML model is fine-tuned using a supervised training corpus of guideline-to-questionnaire mappings curated by clinical domain experts. During fine-tuning, the model learns to recognize patterns of rule-to-question translation, including the framing of temporal constraints (“within the last 90 days”), threshold parameters (e.g., “BMI ≥ 40”), and documentation qualifiers (“documented by a licensed provider”). This training enables the model to generalize across diverse guideline structures while preserving clinical specificity. The output of the second ML model may be a structured questionnaire artifact represented in machine-readable formats, such as HL7 FHIR Questionnaire, JSON, or XML. The system may store the artifact with version control and may digitally sign the artifact for integrity.

### 5. Leveraging Genai to Generate Prefilled questionnaires from Unstructured data for use by Genai in Evaluating Medical necessity of Medical Procedures for Patient conditions

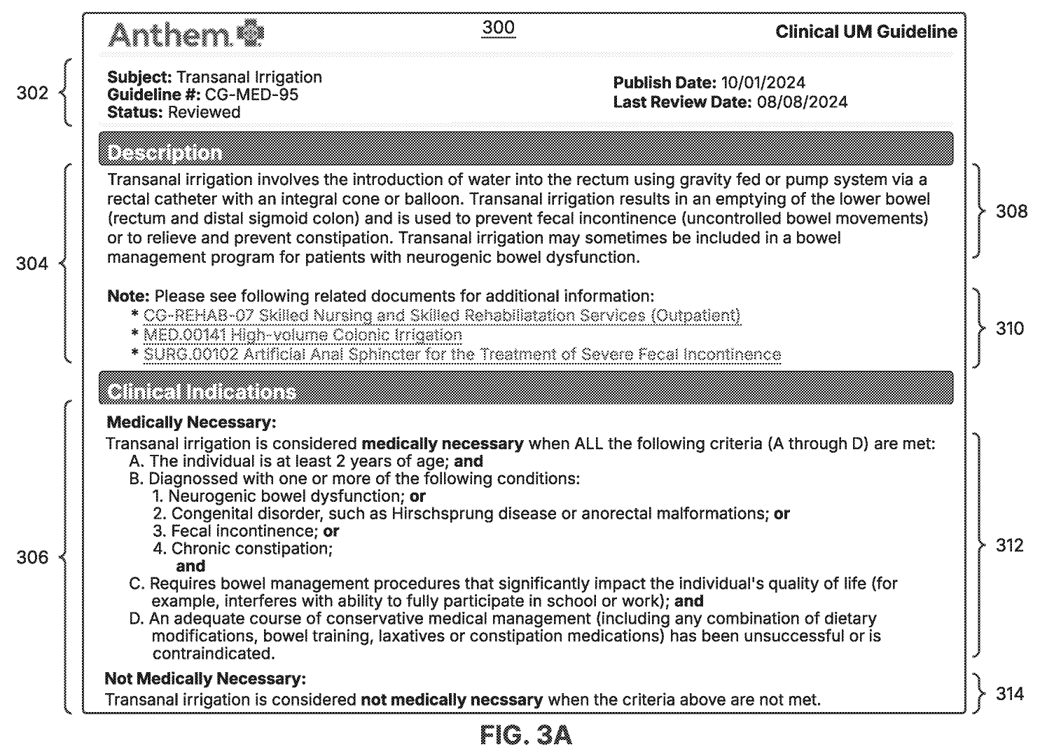

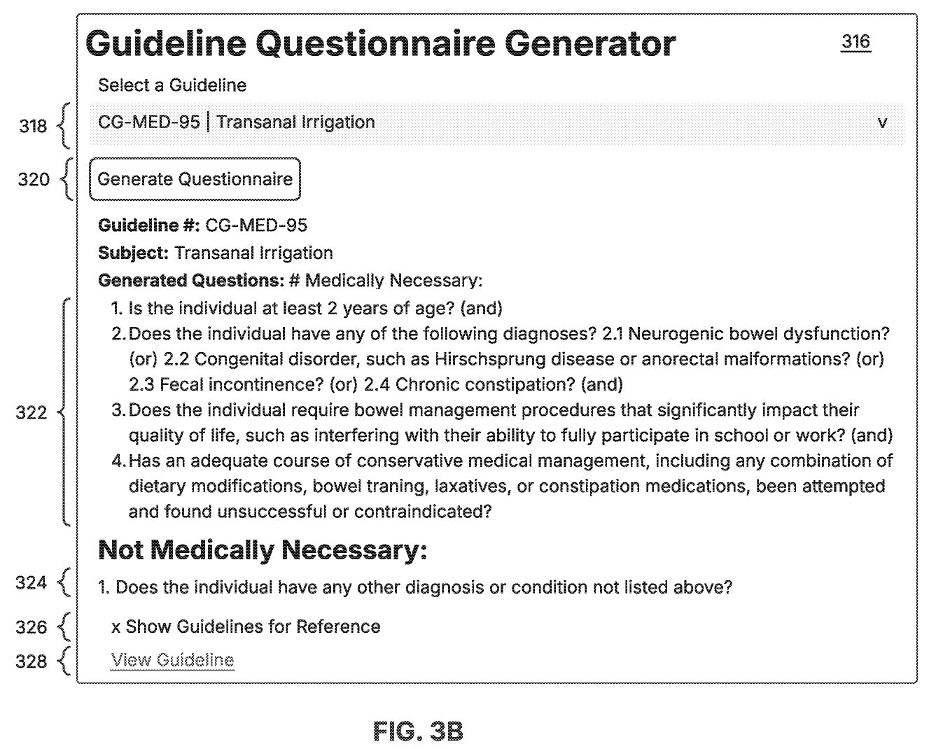

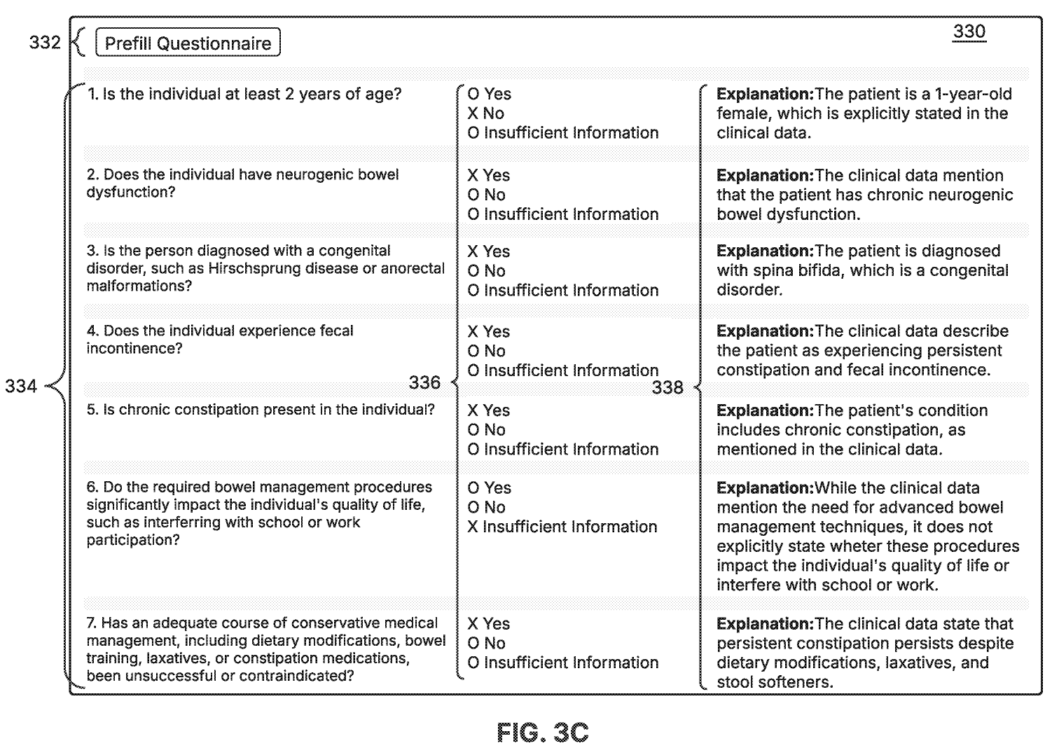

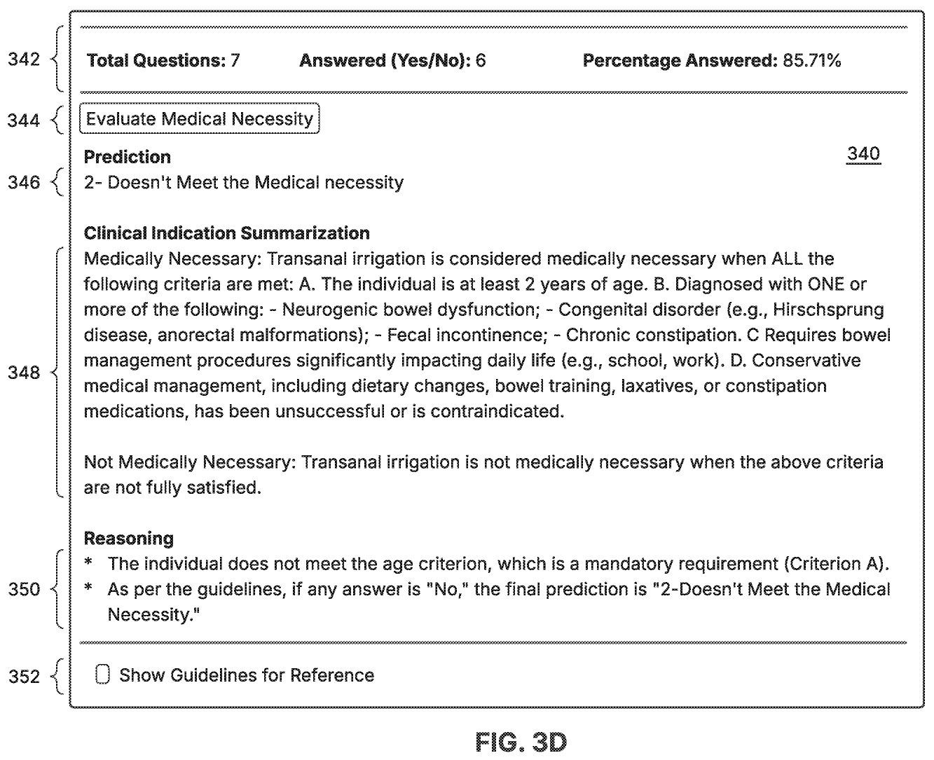

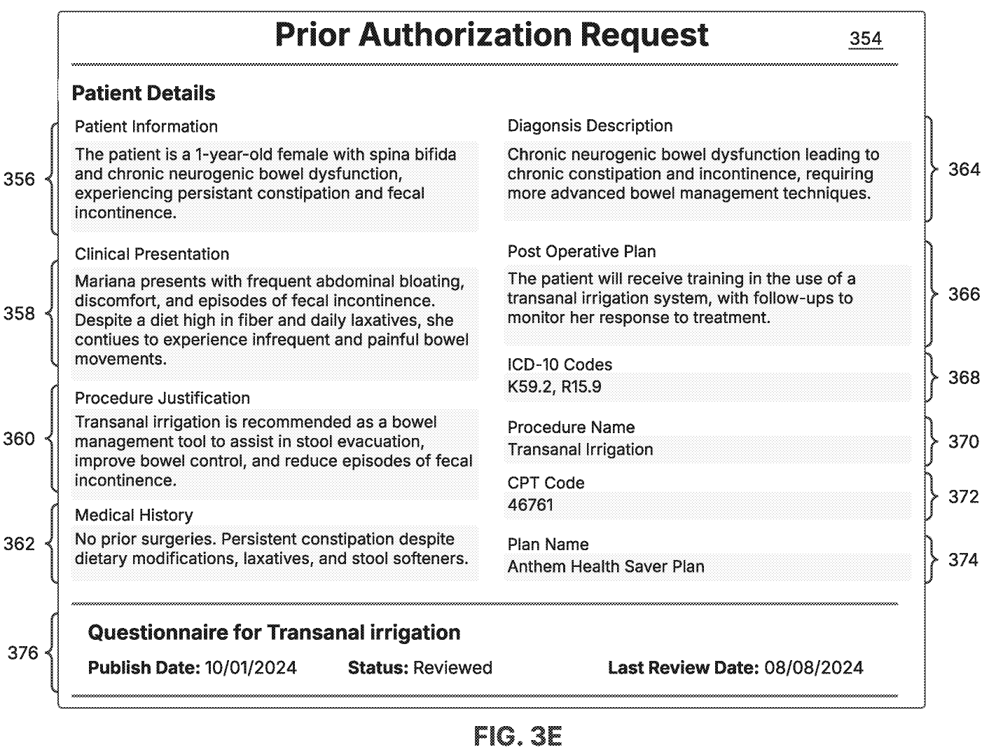

FIG. 2C illustrates an example set of operations for leveraging GenAI to generate prefilled questionnaires for evaluating medical necessity of medical procedures for patient conditions in accordance with one or more embodiments. One or more operations illustrated in FIG. 3C may be modified, rearranged, or omitted. Accordingly, the particular sequence of operations illustrated in FIG. 2C should not be construed as limiting the scope of one or more embodiments.

One or more embodiments parse patient data into a plurality of subsets (Operation 206a). The system may use parsing tools with the patient data to sort the patient data into the plurality of subsets. The system may sort the patient data into various sets and subsets of patient data. The system may sort the patient data by demographic information, clinical information, diagnostic and testing data, treatment and care plans, procedures and interventions, social determinants of health, behavioral and mental health, insurance and financial information, administrative and encounter data, and/or patient-reported outcomes. The system may further sort the patient data into subsets within a set.

In one or more embodiments, python-based parsing tools, such as pdfplumber library, are used to parse the patient data.

One or more embodiments generate a patient data vector embedding for each subset of the plurality of the subsets of patient data (Operation 206b). The system applies a vector embedding function to the text of each subset or chunk of the plurality of subsets of the patient data to generate a vector embedding for each subset. The system may index the vector embeddings for the plurality of subsets of patient data in a vector store.

In one or more embodiments, the text for each subset/chunk is transformed into a high-dimensional vector embedding using Cohere’s LM, e.g., embed-english-v3.0. The system may use domain-specific LMs, e.g., SapBERT, Bio-Clinical BERT, to generate the vector embeddings. The system may use weights to prioritize vector embeddings with greater importance.

In one or more embodiments, text of each subset/chunk of patient data is indexed into a vector store/vector database using Hierarchical Navigable Small World (HNSW). Alternatively, other indexers, such as inverted file index (IVF), can be utilized. Indexing the subsets of patient data in this manner enables efficient handling of the high-dimensional data. Domain-specific LMs and general-purpose LMs may be utilized to encode the semantic meaning of the text. This format is optimized for rapid, similarity-based retrieval in a RAG framework. A vector store, including the patient data vector embeddings, may be saved to disk to ensure easier reload during queries.

One or more embodiments generate a question vector embedding for each question of the clinical guideline questionnaire (Operation 206c). The system applies a vector embedding to the text of each question of the clinical guideline questionnaire to generate question vector embeddings. The system may use the same vector embedding function that was used to generate the patient data vector embeddings to generate the question vector embeddings. Alternatively, the system may use another vector embedding function to generate the question vectors.

One or more embodiments calculate a similarity score for the question vector embedding and the patient data vector embedding (Operation 206d). The system may use the question vector embedding to perform a k-nearest neighbor (k-NN) search against the plurality of patient data vector embeddings stored in the HNSW index. Both the plurality of patient data vector embeddings and the question query vector embedding are loaded into memory. The HNSW index is an optimized data structure that enables fast similarity search. The patient data file may be used for reference to fetch the text associated with the patient data vector embeddings identified as relevant. The k-NN search identifies a top k patient data vector embeddings.

In one or more embodiments, the top k clinical guideline vector embeddings are filtered. Filtering the top k patient vector embeddings may include determining a similarity score for each of the top k patient data vector embeddings and the question vector embedding. The similarity score may be calculated using cosine similarity.

One or more embodiments determine that the similarity score for the question vector embedding and the patient data vector embedding exceeds a threshold value for including the subset of patient data in a set of patient data for answering the question (Operation 206e). Similarity scores range from 0 – 1.0. The closer to 1.0 the similarity score, the greater the similarity between the clinical guideline vector embedding and the request query vector embedding. The threshold value for the similarity may be determined by the user. In an example, the threshold value is 0.5. In this manner, each subset of patient data that is associated with a patient data vector embedding in the top k patient data vector embeddings and has a similarity score of 0.5 or higher with the question vector embedding is deemed relevant.

If the similarity score for a question vector embedding and a patient data vector embedding does not exceed threshold value, one or more embodiments exclude the subset of patient data from the set of patient data for answering the question (Operation 206f). The system may exclude top k patient data vector embeddings with a similarity score under a threshold score, e.g., 0.5. In this manner, each subset of patient data that is associated with a patient data vector embedding in the top k patient data vector embeddings and has a similarity score below 0.5 with the question vector embedding is filtered from the set of relevant subsets of patient data to create a set of relevant subsets of patient data.

In one or more embodiments, the system applies a reranking model to the subsets of patient data determined to be relevant to rerank the relevant subsets of patient data and identify the most relevant subsets of patient data. More particularly, the system applies reranking model to the set of relevant subsets of patient data. The reranking model may evaluate the subsets of patient data using multiple fields, including, for example, conditions, treatments, and/or dates of treatment, to ensure that best matches are reranked highest. The reranking model refines and reorders the relevant subsets of patient data based on relevance by evaluating the semantic relationship between the results and the questions.

When the similarity score for a question vector embedding and a patient data vector embedding does exceed threshold value, one or more embodiments provide, to a third ML model, the subset(s) of patient data for use in answering the particular question (Operation 206g). The system may prompt the second ML model to answer the questions of the clinical guideline question. An example LLM includes GPT4Turbo. The system may use short learning to train or fine-tune the ML model used to determine the answers to the questionnaire. The system may automatically identify the subsets of patient data associated with the top reranked patient data vector embeddings as the most relevant patient data or target patient data for answering the particular question. The system may then provide the most relevant patient data for answering the particular question to the ML model to generate an answer for the particular question.

One or more embodiments populate a question field for the question of the clinical guideline questionnaire with the subset of patient data (Operation 206h). The GenAI model uses the proffered subsets of patient data to generate answers for the particular questions. The system may populate the question field of the particular question with the answer to the question generated by the ML model.

### 6. Example Embodiments

A detailed example is described below for purposes of clarity. Components and/or operations described below should be understood as one specific example which may not be applicable to certain embodiments. Accordingly, components and/or operations described below should not be construed as limiting the scope of any of the claims.

FIG. 3A illustrates an example clinical guideline in accordance with one or more embodiments. Clinical guideline 300 is provided by Anthem Healthcare. The system may scrape clinical guideline 300 from Anthem Healthcare website, or download clinical guideline 300 through an API provided by Anthem Healthcare, or access the clinical guideline 300 via any other suitable manner. Clinical guideline 300 includes a header section 302, a description section 304, and a clinical indications section 306.

Header section 302 includes details for the clinical guideline 300, including a subject, a guideline number, status, a publish date, and a last review date. The subject includes the particular medical procedure identified in clinical guideline 300, i.e., transanal irrigation. The guideline number provides a reference code for the particular medical procedure identified in clinical guideline 300. The status is an indication of the status of clinical guideline 300, e.g., reviewed. The publish date is the date that clinical guideline 300 was published. The last review date is the date that clinical guideline 300 was most recently reviewed.

Description section 304 of clinical guideline 300 includes a description 308 and related documents 310. Description 308 is a narrative summary of the particular medical procedure identified in clinical guideline 300. Related documents 310 is a list of documents related to the medical procedure identified in the clinical guideline 300. The list of related documents may be provided as links to the relevant documents.

Clinical indications section 306 is divided into a medically necessary section 312 and a not medically necessary section 314. Medically necessary section 312 includes one or more conditions that a patient is required to satisfy for the particular medical procedure identified in clinical guideline 300 to be considered medically necessary for the patient. Not medically necessary section 314 includes one or more conditions that disqualify the procedure from being medically necessary.

FIG. 3B illustrates an example clinical guideline questionnaire generator interface in accordance with one or more embodiments. A clinical guideline questionnaire generator automatically ingests the clinical guideline 300 (FIG. 3A) and parses the clinical guideline 300 to identify the particular medical procedure identified in the clinical guideline and the patient conditions required for determining medical necessity of the procedure for the patient. A clinical guideline questionnaire generator interface 316 includes a guideline selection field 318 for selecting a clinical guideline/medical procedures from a list of clinical guidelines/medical procedures for generating a questionnaire for the clinical guideline/medical procedure. As shown, guideline selection field 318 is a pulldown menu.

After selecting a clinical guideline/medical procedure, e.g., CG-MED-95 | Transanal Irrigation, a clinician may engage a first interface element 320 of the clinical guideline questionnaire generator interface 316 to generate a clinical guideline questionnaire. Initially, the guideline questionnaire generator ingests the particular clinical guideline, e.g., clinical guideline 300 (FIG. 3A). The guideline questionnaire generator then applies a first GenAI model to the ingested clinical guideline to generate one or more questions 322 for determining that the medical procedure is medical necessary. The guideline questionnaire generator also applies the first GenAI model to the ingested clinical guideline to generate one or more questions 324 for determining that the medical procedure is not medically necessary. Clinical guideline questionnaire generator interface 316 may include a second interface element 326 for selecting a showing of the particular clinical guideline for reference as well as a third interface element 328 for linking to the clinical guideline 300.

FIG. 3C illustrates an example of a prefilled questionnaire interface in accordance with one or more embodiments. Prefilled questionnaire interface 330 includes an interface element 332 for generating a prefilled questionnaire. When a clinician engages interface element 332, the system applies a second GenAI model to the clinical guideline questionnaire generated by the clinical guideline questionnaire generator for the particular medical procedure. The system also applies the second GenAI model to a set of patient data for the patient. The system generates a prefilled questionnaire that includes questions 334 for determining medical necessity of the procedure, answers 336 to questions 334, and explanations 338 for answers 336.

FIG. 3D illustrates an example evaluation interface with reasoning in accordance with one or more embodiments. Evaluation interface 340 includes a questionnaire summary section 342 and a first interface element 344 for generating an evaluation of medical necessity. Questionnaire summary section 342 includes total number of questions in a particular prefilled clinical guideline questionnaire, number of questions answered in the particular prefilled guideline questionnaire, and percentage of the questions in the particular prefilled guideline questionnaire that are answered.

When a clinician engages first interface element 344, the system applies a third GenAI model to the particular prefilled guideline questionnaire to generate an evaluation of medical necessity of the particular medical procedure for the patient. The evaluation includes a prediction 346, a clinical indication summary 348, and reasoning 350. Prediction 346 indicates if the patient conditions meet the conditions for medical necessity of the particular medical procedure. Clinical indication summary 348 details the conditions required for medical necessity of the medical procedure, and clinical indication summary 348 also details the conditions when a medical procedure is not medically necessary. Reasoning 350 provides an explanation of the patient conditions that caused a determination that the medical procedure is medically necessary or is not medically necessary. Evaluation interface 340 may include a second interface element 352 for providing the particular guideline for reference.

FIG. 3E illustrates an example prior authorization request interface for a medical procedure in accordance with one or more embodiments. Prior authorization interface 354 provides numerous patient details, including patient information 356, clinical presentation 358, procedure justification 360, medical history 362, diagnosis description 364, post operative plan 366, ICD-10 codes 368, procedure name 370, CPT codes 372, and plan name 374. Prior authorization interface 354 may further include a status summary 376 of the particular clinical guideline, e.g., clinical guideline 300, used to generate the clinical guideline questionnaire. The system may automatically submit the prior authorization request to a third-party payer for review and approval.

### 7. Machine Learning Architecture

FIG. 4A illustrates a machine learning engine 400 in accordance with one or more embodiments. As illustrated in FIG. 4A, machine learning engine 400 includes input/output module 402, data preprocessing module 404, model selection module 406, training module 408, evaluation and tuning module 410, and inference module 412.

In accordance with an embodiment, input/output module 402 serves as the primary interface for data entering and exiting the system, managing the flow and integrity of data. This module may accommodate a wide range of data sources and formats to facilitate integration and communication within the machine learning architecture.

In an embodiment, an input handler within input/output module 402 includes a data ingestion framework capable of interfacing with various data sources, such as databases, APIs, file systems, and real-time data streams. This framework is equipped with functionalities to handle different data formats (e.g., CSV, JSON, XML) and efficiently manage large volumes of data. It includes mechanisms for batch and real-time data processing that enable the input/output module 402 to be versatile in different operational contexts, whether processing historical datasets or streaming data.

In accordance with an embodiment, input/output module 402 manages data integrity and quality as it enters the system by incorporating initial checks and validations. These checks and validations ensure that incoming data meets predefined quality standards, like checking for missing values, ensuring consistency in data formats, and verifying data ranges and types. This proactive approach to data quality minimizes potential errors and inconsistencies in later stages of the machine learning process.

In an embodiment, an output handler within input/output module 402 includes an output framework designed to handle the distribution and exportation of outputs, predictions, or insights. Using the output framework, input/output module 402 formats these outputs into user-friendly and accessible formats, such as reports, visualizations, or data files compatible with other systems. Input/output module 402 also ensures secure and efficient transmission of these outputs to end-users or other systems in an embodiment and may employ encryption and secure data transfer protocols to maintain data confidentiality.

In accordance with an embodiment, data preprocessing module 404 transforms data into a format suitable for use by other modules in machine learning engine 400. For example, data preprocessing module 404 may transform raw data into a normalized or standardized format suitable for training ML models and for processing new data inputs for inference. In an embodiment, data preprocessing module 404 acts as a bridge between the raw data sources and the analytical capabilities of machine learning engine 400.

In an embodiment, data preprocessing module 404 begins by implementing a series of preprocessing steps to clean, normalize, and/or standardize the data. This involves handling a variety of anomalies, such as managing unexpected data elements, recognizing inconsistencies, or dealing with missing values. Some of these anomalies can be addressed through methods like imputation or removal of incomplete records, depending on the nature and volume of the missing data. Data preprocessing module 404 may be configured to handle anomalies in different ways depending on context. Data preprocessing module 404 also handles the normalization of numerical data in preparation for use with models sensitive to the scale of the data, like neural networks and distance-based algorithms. Normalization techniques, such as min-max scaling or z-score standardization, may be applied to bring numerical features to a common scale, enhancing the model’s ability to learn effectively.

In an embodiment, data preprocessing module 404 includes a feature encoding framework that ensures categorical variables are transformed into a format that can be easily interpreted by machine learning algorithms. Techniques like one-hot encoding or label encoding may be employed to convert categorical data into numerical values, making them suitable for analysis. The module may also include feature selection mechanisms, where redundant or irrelevant features are identified and removed, thereby increasing the efficiency and performance of the model.

In accordance with an embodiment, when data preprocessing module 404 processes new data for inference, data preprocessing module 404 replicates the same preprocessing steps to ensure consistency with the training data format. This helps to avoid discrepancies between the training data format and the inference data format, thereby reducing the likelihood of inaccurate or invalid model predictions.

In an embodiment, model selection module 406 includes logic for determining the most suitable algorithm or model architecture for a given dataset and problem. This module operates in part by analyzing the characteristics of the input data, such as its dimensionality, distribution, and the type of problem (classification, regression, clustering, etc.).

In an embodiment, model selection module 406 employs a variety of statistical and analytical techniques to understand data patterns, identify potential correlations, and assess the complexity of the task. Based on this analysis, it then matches the data characteristics with the strengths and weaknesses of various available models. This can range from simple linear models for less complex problems to sophisticated deep learning architectures for tasks requiring feature extraction and high-level pattern recognition, such as image and speech recognition.

In an embodiment, model selection module 406 utilizes techniques from the field of Automated Machine Learning (AutoML). AutoML systems automate the process of model selection by rapidly prototyping and evaluating multiple models. They use techniques like Bayesian optimization, genetic algorithms, or reinforcement learning to explore the model space efficiently. Model selection module 406 may use these techniques to evaluate each candidate model based on performance metrics relevant to the task. For example, accuracy, precision, recall, or F1 score may be used for classification tasks and mean squared error metrics may be used for regression tasks. Accuracy measures the proportion of correct predictions (both positive and negative). Precision measures the proportion of actual positives among the predicted positive cases. Recall (also known as sensitivity) evaluates how well the model identifies actual positives. F1 Score is a single metric that accounts for both false positives and false negatives. The mean squared error (MSE) metric may be used for regression tasks. MSE measures the average squared difference between the actual and predicted values, providing an indication of the model’s accuracy. A lower MSE may indicate a model’s greater accuracy in predicting values, as it represents a smaller average discrepancy between the actual and predicted values.

In accordance with an embodiment, model selection module 406 also considers computational efficiency and resource constraints. This is meant to help ensure the selected model is both accurate and practical in terms of computational and time requirements. In an embodiment, certain features of model selection module 406 are configurable such as a configured bias toward (or against) computational efficiency.

In accordance with an embodiment, training module 408 manages the ‘learning’ process of ML models by implementing various learning algorithms that enable models to identify patterns and make predictions or decisions based on input data. In an embodiment, the training process begins with the preparation of the dataset after preprocessing; this involves splitting the data into training and validation sets. The training set is used to teach the model, while the validation set is used to evaluate its performance and adjust parameters accordingly. Training module 408 handles the iterative process of feeding the training data into the model, adjusting the model’s internal parameters (like weights in neural networks) through backpropagation and optimization algorithms, such as stochastic gradient descent or other algorithms providing similarly useful results.

In accordance with an embodiment, training module 408 manages overfitting, where a model learns the training data too well, including its noise and outliers, at the expense of its ability to generalize to new data. Techniques such as regularization, dropout (in neural networks), and early stopping are implemented to mitigate this. Additionally, the module employs various techniques for hyperparameter tuning; this involves adjusting model parameters that are not directly learned from the training process, such as learning rate, the number of layers in a neural network, or the number of trees in a random forest.

In an embodiment, training module 408 includes logic to handle different types of data and learning tasks. For instance, it includes different training routines for supervised learning (where the training data comes with labels) and unsupervised learning (without labeled data). In the case of deep learning models, training module 408 also manages the complexities of training neural networks that include initializing network weights, choosing activation functions, and setting up neural network layers.

In an embodiment, evaluation and tuning module 410 incorporates dynamic feedback mechanisms and facilitates continuous model evolution to help ensure the system’s relevance and accuracy as the data landscape changes. Evaluation and tuning module 410 conducts a detailed evaluation of a model’s performance. This process involves using statistical methods and a variety of performance metrics to analyze the model’s predictions against a validation dataset. The validation dataset, distinct from the training set, is instrumental in assessing the model’s predictive accuracy and its capacity to generalize beyond the training data. The module’s algorithms meticulously dissect the model’s output, uncovering biases, variances, and the overall effectiveness of the model in capturing the underlying patterns of the data.

In an embodiment, evaluation and tuning module 410 performs continuous model tuning by using hyperparameter optimization. Evaluation and tuning module 410 performs an exploration of the hyperparameter space using algorithms, such as grid search, random search, or more sophisticated methods like Bayesian optimization. Evaluation and tuning module 410 uses these algorithms to iteratively adjust and refine the model’s hyperparameters – settings that govern the model’s learning process but are not directly learned from the data – to enhance the model’s performance. This tuning process helps to balance the model’s complexity with its ability to generalize and attempts to avoid the pitfalls of underfitting or overfitting.

In an embodiment, evaluation and tuning module 410 integrates data feedback and updates the model. Evaluation and tuning module 410 actively collects feedback from the model’s real-world applications, an indicator of the model’s performance in practical scenarios. Such feedback can come from various sources depending on the nature of the application. For example, in a user-centric application like a recommendation system, feedback might comprise user interactions, preferences, and responses. In other contexts, such as predicting events, it might involve analyzing the model’s prediction errors, misclassifications, or other performance metrics in live environments.

In an embodiment, feedback integration logic within evaluation and tuning module 410 integrates this feedback using a process of assimilating new data patterns, user interactions, and error trends into the system’s knowledge base. The feedback integration logic uses this information to identify shifts in data trends or emergent patterns that were not present or inadequately represented in the original training dataset. Based on this analysis, the module triggers a retraining or updating cycle for the model. If the feedback suggests minor deviations or incremental changes in data patterns, the feedback integration logic may employ incremental learning strategies, fine-tuning the model with the new data while retaining its previously learned knowledge. In cases where the feedback indicates significant shifts or the emergence of new patterns, a more comprehensive model updating process may be initiated. This process might involve revisiting the model selection process, re-evaluating the suitability of the current model architecture, and/or potentially exploring alternative models or configurations that are more attuned to the new data.

In accordance with an embodiment, throughout this iterative process of feedback integration and model updating, evaluation and tuning module 410 employs version control mechanisms to track changes, modifications, and the evolution of the model, facilitating transparency and allowing for rollback if necessary. This continuous learning and adaptation cycle, driven by real-world data and feedback, helps to endure the model’s ongoing effectiveness, relevance, and accuracy.

In an embodiment, inference module 412 transforms data raw data into actionable, precise, and contextually relevant predictions. In addition to processing and applying a trained model to new data, inference module 412 may also include post-processing logic that refines the raw outputs of the model into meaningful insights.

In an embodiment, inference module 412 includes classification logic that takes the probabilistic outputs of the model and converts them into definitive class labels. This process involves an analytical interpretation of the probability distribution for each class. For example, in binary classification, the classification logic may identify the class with a probability above a certain threshold, but classification logic may also consider the relative probability distribution between classes to create a more nuanced and accurate classification.

In an embodiment, inference module 412 transforms the outputs of a trained model into definitive classifications. Inference module 412 employs the underlying model as a tool to generate probabilistic outputs for each potential class. It then engages in an interpretative process to convert these probabilities into concrete class labels.

In an embodiment, when inference module 412 receives the probabilistic outputs from the model, it analyzes these probabilities to determine how they are distributed across some or every potential class. If the highest probability is not significantly greater than the others, inference module 412 may determine that there is ambiguity or interpret this as a lack of confidence displayed by the model.

In an embodiment, inference module 412 uses thresholding techniques for applications where making a definitive decision based on the highest probability might not suffice due to the critical nature of the decision. In such cases, inference module 412 assesses if the highest probability surpasses a certain confidence threshold that is predetermined based on the specific requirements of the application. If the probabilities do not meet this threshold, inference module 412 may flag the result as uncertain or defer the decision to a human expert. Inference module 412 dynamically adjusts the decision thresholds based on the sensitivity and specificity requirements of the application, subject to calibration for balancing the trade-offs between false positives and false negatives.

In accordance with an embodiment, inference module 412 contextualizes the probability distribution against the backdrop of the specific application. This involves a comparative analysis, especially in instances where multiple classes have similar probability scores, to deduce the most plausible classification. In an embodiment, inference module 412 may incorporate additional decision-making rules or contextual information to guide this analysis, ensuring that the classification aligns with the practical and contextual nuances of the application.

In regression models, where the outputs are continuous values, inference module 412 may engage in a detailed scaling process in an embodiment. Outputs, often normalized or standardized during training for optimal model performance, are rescaled back to their original range. This rescaling involves recalibration of the output values using the original data’s statistical parameters, such as mean and standard deviation, ensuring that the predictions are meaningful and comparable to the real-world scales they represent.

In an embodiment, inference module 412 incorporates domain-specific adjustments into its post-processing routine. This involves tailoring the model’s output to align with specific industry knowledge or contextual information. For example, in financial forecasting, inference module 412 may adjust predictions based on current market trends, economic indicators, or recent significant events, ensuring that the outputs are both statistically accurate and practically relevant.

In an embodiment, inference module 412 includes logic to handle uncertainty and ambiguity in the model’s predictions. In cases where inference module 412 outputs a measure of uncertainty, such as in Bayesian inference models, inference module 412 interprets these uncertainty measures by converting probabilistic distributions or confidence intervals into a format that can be easily understood and acted upon. This provides users with both a prediction and an insight into the confidence level of that prediction. In an embodiment, inference module 412 includes mechanisms for involving human oversight or integrating the instance into a feedback loop for subsequent analysis and model refinement.

In an embodiment, inference module 412 formats the final predictions for end-user consumption. Predictions are converted into visualizations, user-friendly reports, or interactive interfaces. In some systems, like recommendation engines, inference module 412 also integrates feedback mechanisms, where user responses to the predictions are used to continually refine and improve the model, creating a dynamic, self-improving system.

### 8. Machine Learning Engine Operations

FIG. 4B illustrates the operation of a machine learning engine in one or more embodiments. In an embodiment, input/output module 402 receives a dataset intended for training (Operation 401). This data can originate from diverse sources, like databases or real-time data streams, and in varied formats, such as CSV, JSON, or XML. Input/output module 402 assesses and validates the data, ensuring its integrity by checking for consistency, data ranges, and types.

In an embodiment, training data is passed to data preprocessing module 404. Here, the data undergoes a series of transformations to standardize and clean it, making it suitable for training ML models (Operation 403). This involves normalizing numerical data, encoding categorical variables, and handling missing values through techniques like imputation.

In an embodiment, prepared data from the data preprocessing module 404 is then fed into model selection module 406 (Operation 405). This module analyzes the characteristics of the processed data, such as dimensionality and distribution, and selects the most appropriate model architecture for the given dataset and problem. It employs statistical and analytical techniques to match the data with an optimal model, ranging from simpler models for less complex tasks to more advanced architectures for intricate tasks.

In an embodiment, training module 408 trains the selected model with the prepared dataset (Operation 407). It implements learning algorithms to adjust the model’s internal parameters, optimizing them to identify patterns and relationships in the training data. Training module 408 also addresses the challenge of overfitting by implementing techniques, like regularization and early stopping, ensuring the model’s generalizability.

In an embodiment, evaluation and tuning module 410 evaluates the trained model’s performance using the validation dataset (Operation 409). Evaluation and tuning module 410 applies various metrics to assess predictive accuracy and generalization capabilities. It then tunes the model by adjusting hyperparameters, and if needed, incorporates feedback from the model’s initial deployments, retraining the model with new data patterns identified from the feedback.

In an embodiment, input/output module 402 receives a dataset intended for inference. Input/output module 402 assesses and validates the data (Operation 411).

In an embodiment, data preprocessing module 404 receives the validated dataset intended for inference (Operation 413). Data preprocessing module 404 ensures that the data format used in training is replicated for the new inference data, maintaining consistency and accuracy for the model’s predictions.

In an embodiment, inference module 412 processes the new data set intended for inference, using the trained and tuned model (Operation 415). It applies the model to this data, generating raw probabilistic outputs for predictions. Inference module 412 then executes a series of post-processing steps on these outputs, such as converting probabilities to class labels in classification tasks or rescaling values in regression tasks. It contextualizes the outputs as per the application’s requirements, handling any uncertainty in predictions and formatting the final outputs for end-user consumption or integration into larger systems.

In an embodiment, machine learning engine API 414 allows for applications to leverage machine learning engine 400. In an embodiment, machine learning engine API 414 may be built on a RESTful architecture and offer stateless interactions over standard HTTP/HTTPS protocols. Machine learning engine API 414 may feature a variety of endpoints, each tailored to a specific function within machine learning engine 400. In an embodiment, endpoints such as /submitData facilitate the submission of new data for processing, while /retrieveResults is designed for fetching the outcomes of data analysis or model predictions. The MLE API may also include endpoints like /updateModel for model modifications and /trainModel to initiate training with new datasets.

In an embodiment, machine learning engine API 414 is equipped to support SOAP-based interactions. This extension involves defining a WSDL (Web Services Description Language) document that outlines the API’s operations and the structure of request and response messages. In an embodiment, machine learning engine API 414 supports various data formats and communication styles. In an embodiment, machine learning engine API 414 endpoints may handle requests in JSON format or any other suitable format. For example, machine learning engine API 414 may process XML, and it may also be engineered to handle more compact and efficient data formats, such as Protocol Buffers or Avro, for use in bandwidth-limited scenarios.

In an embodiment, machine learning engine API 414 is designed to integrate WebSocket technology for applications necessitating real-time data processing and immediate feedback. This integration enables a continuous, bi-directional communication channel for a dynamic and interactive data exchange between the application and machine learning engine 400.

### 9. Generative AI Models

A generative AI model is a machine learning model that is capable of generating new data instances based on the data used to train the model. A generative model may be referred to as a “generative artificial intelligence (AI) model.” Generative models learn the underlying distribution of the training data, enabling them to produce new instances of data that share properties with the original dataset. This capability makes them particularly useful in a variety of applications, including image and voice generation, text synthesis, and more sophisticated tasks like unsupervised learning, semi-supervised learning, and domain adaptation.

One type of generative model is a large language model. Large language models are designed to understand, generate, and interpret human language by processing extensive collections of data. The foundational architecture behind large language models is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times.

In an embodiment, a mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

In accordance with one or more embodiments, transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a SoftMax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

In accordance with one or more embodiments, following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model’s hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

In accordance with one or more embodiments, integrating these components within the transformer architecture facilitates the model’s ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

In accordance with one or more embodiments, input/output module 402, when used for large language models, handles textual data, converting input text into a format that the model can process. This typically involves tokenization, where the text is broken down into manageable pieces, such as words or sub words, and then converted into numerical representations. These representations, or embeddings, capture semantic information about the text that is then fed into the model for processing. The output from the model is converted from numerical form back into human-readable text, following the generation of predictions or responses.

In accordance with one or more embodiments, data preprocessing module 404 in the context of large language models may include steps such as normalization, where the text is converted to a uniform case and punctuation is standardized. This process ensures that the model treats similar words or symbols consistently, reducing the complexity of the input space. Additionally, techniques such as sentence segmentation may be applied to manage longer texts, enabling the model to process information in chunks that align with natural language structures.

In accordance with one or more embodiments, model selection module 406, when used for large language models involves choosing a specific architecture and configuration that is best suited to the task at hand. This decision is based on various factors, such as the size of the available training data, the complexity of the language tasks to be performed, and computational resource constraints. Models may vary in size from millions to billions of parameters, with larger models generally capable of more nuanced language understanding and generation but requiring significantly more computational power to train and operate.

In accordance with one or more embodiments, training module 408, when used for large language models, is configured to adjust the model’s parameters through exposure to training data. This process utilizes optimization algorithms, such as stochastic gradient descent, to minimize the difference between the model’s predictions and the actual desired outputs. The training process is computationally intensive, often requiring specialized hardware such as GPUs (Graphics Processing Units) or TPUs (Tensor Processing Units) to manage the large volumes of data and the complexity of the model calculations. During training, techniques, such as dropout and layer normalization, are used to improve model generalization and prevent overfitting (i.e., when a model learns the detail and noise in the training data to the extent that it negatively impacts the model’s performance on new data).

In accordance with one or more embodiments, evaluation and tuning module 410 assesses the performance of large language models using metrics such as perplexity, accuracy, and F1 score, depending on the specific language tasks. Evaluation may involve comparing the model’s output against a set of labeled validation data, providing insight into how well the model has learned to perform tasks, such as text classification, question answering, or text generation. Tuning involves adjusting model parameters or training strategies based on evaluation outcomes to improve performance. This may include hyperparameter tuning, where parameters that govern the training process, such as learning rate or batch size, are adjusted.

In accordance with one or more embodiments, inference module 412, in the context of large language models, is responsible for generating predictions or responses based on new, unseen data. This process involves feeding the input data through the trained model to produce an output. Inference can be used for a variety of applications, including translating text, generating human-like responses in a chatbot, or summarizing articles.

Another type of generative model is a large multimodal model (LMM). A large multimodal model is an advanced machine learning model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

The self-attention mechanism, which is part of a transformer network, enables the model to weigh the importance of different elements within an input sequence, regardless of their position. This allows the model to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing is a step that ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model’s parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model’s effectiveness. This iterative process ensures that the model can perform a wide range of multimodal tasks with high accuracy and relevance, making it a versatile tool for applications requiring the integration of different types of data.

Large multimodal models represent a significant advancement in machine learning by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides large language models and large multimodal models belong to the broad category of generative models. For example, stochastic models directly incorporate randomness into their structure, making them inherently generative as they can produce a diverse set of outputs for a given input. Generative Adversarial Networks (GANs) learn to generate new data that is indistinguishable from the data they were trained on, using a dual-network architecture that involves a generative component. Variational Autoencoders (VAEs) are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond large language models.

Although generative models can be leveraged for classification tasks, they inherently operate on principles of randomness, leading to a spectrum of possible outcomes in response to identical inputs. Unlike deterministic models that yield a consistent result whenever the same input is given, generative models use the randomness in the data they are trained on to both mimic and diversify from the training data. This diversity makes generative models ideal for generating new and varied data points as well as for tasks that require creativity and novelty. However, a reliance on randomness creates a trade-off between predictability and flexibility for generative models, potentially making them less predictable in scenarios where uniform outcomes may be expected such as classification tasks.

### 10. Practical Applications, Advantages & Improvements

Embodiments provide several practical applications, advantages, and improvements over existing manual and rule-based prior authorization systems. These advantages and improvements include the ability to automatically interpret clinical guidelines, dynamically generate and prefill medical necessity questionnaires, and produce explainable determinations of coverage eligibility in real time.

Embodiments utilize a multi-layer architecture integrating GenAI, vector embeddings, similarity scoring, and adaptive feedback mechanisms to transform fragmented patient data into structured, evidence-backed authorization requests. By bridging patient data, clinical guideline rules, and payer policy frameworks through ML, the disclosed system automates complex medical necessity evaluations that traditionally require extensive human review.

Embodiments improve accuracy in medical necessity determination by applying GenAI-driven reasoning in conjunction with vector embeddings and similarity scores to semantically align patient-specific clinical evidence with clinical guideline criteria. For example, when evaluating if a patient has met prerequisite conservative management requirements, the system compares EHR-derived data embeddings against guideline embeddings and generates a similarity score validated by threshold scores. This ensures consistent, evidence-based determinations aligned with current payer and clinical standards.

Embodiments significantly reduce administrative burden and processing time by automatically generating and pre-filling questionnaires based on patient data parsed from EHR and other sources. This results in faster authorization cycles, fewer incomplete submissions, and improved scalability across multiple payers and procedures. For instance, the questionnaire prefill module populates each question with relevant patient data and explicit justifications, enabling same-session authorization readiness without manual data entry.

Embodiments enhance transparency by generating explainable evaluations that include structured evidence rationales and provenance metadata, linking each authorization decision to the originating patient data, guideline rule, and model configuration. For instance, if a denial is predicted, the system provides a rationale, such as, “Criterion A not satisfied—patient age (1 year) below required minimum (2 years),” ensuring human-reviewable justification for every automated decision.

Embodiments improve upon existing approaches by reducing authorization errors and payer-provider rework by converting unstructured clinical narratives into semantically normalized evidence aligned with guideline logic. This reduces manual misinterpretations and inconsistent decision outcomes.

Embodiments provide a technical benefit by transforming patient data into machine-interpretable embeddings, enabling cross-model semantic matching between patient information and clinical guideline rules. This results in deterministic, traceable, and reproducible decision outputs that improve model explainability and audit compliance. The transformation is accomplished through integrated NLP parsing, embedding computation, and threshold-based scoring pipelines.

Embodiments implementing a GenAI-powered prior authorization assistant, as described herein, enable real-time, AI-assisted evaluation of medical necessity. The system automatically constructs questionnaires, prefills patient-specific responses, and computes evaluation scores in compliance with payer-specific coverage policies. This is accomplished through modular orchestration of a questionnaire generator module, questionnaire prefill module, and an evaluation module.

One or more embodiments improve the functioning of computer systems in the technical field of healthcare data interoperability by enabling the automated ingestion, normalization, and reasoning over heterogeneous patient and policy data. This improvement provides both technical and economic benefits by reducing latency in payer decision-making and eliminating redundant manual review steps. This practical application distinguishes existing approaches by providing an explainable, continuously learning AI workflow integrated with real-time guideline updates and payer feedback.

In an embodiment, the system improves medical necessity determinations by employing a dual-inference architecture that combines deterministic rule execution with GenAI-driven contextual reasoning. This improvement ensures that even ambiguous or narrative clinical statements are correctly mapped to structured decision criteria. For example, a provider note indicating that “symptoms persist despite diet and medication” may be automatically recognized as satisfying the “failed conservative therapy” criterion, reducing manual reviewer intervention.

By providing adaptive, explainable, and interoperable automation of prior authorization workflows, embodiments address a longstanding problem in healthcare administration – the latency and subjectivity of manual medical necessity reviews. This results in faster, more consistent, and evidence-based determinations that improve provider efficiency, payer accuracy, and patient access to timely care. The system accomplishes this by integrating GenAI-driven language understanding, embedding-based evidence alignment, and feedback-informed model refinement within a unified decision-support architecture.

The data input to any ML model and/or the data output from any ML model, as described herein, may be used for operations performed by one or more of the following types of software: Database, Cloud Infrastructure, Customer Relationship Management, Data Science, Digital Assistant, Vision, Language, Speech, Forecasting, Enterprise, Middleware, Server, Identity Management, Application Development, Analytics, Security, Data Integration, Health, Hospitality, Retail, Utilities, Operating Systems, Virtualization, Governance and Administration, Migration & Disaster Recovery, Networking, Connectivity, Monitoring, Procurement, Project Management, Risk Management, Supply Chain Management, Manufacturing, Human Capital Management, Customer Experience, Advertising, and Industry-Specific Application.

### 11. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

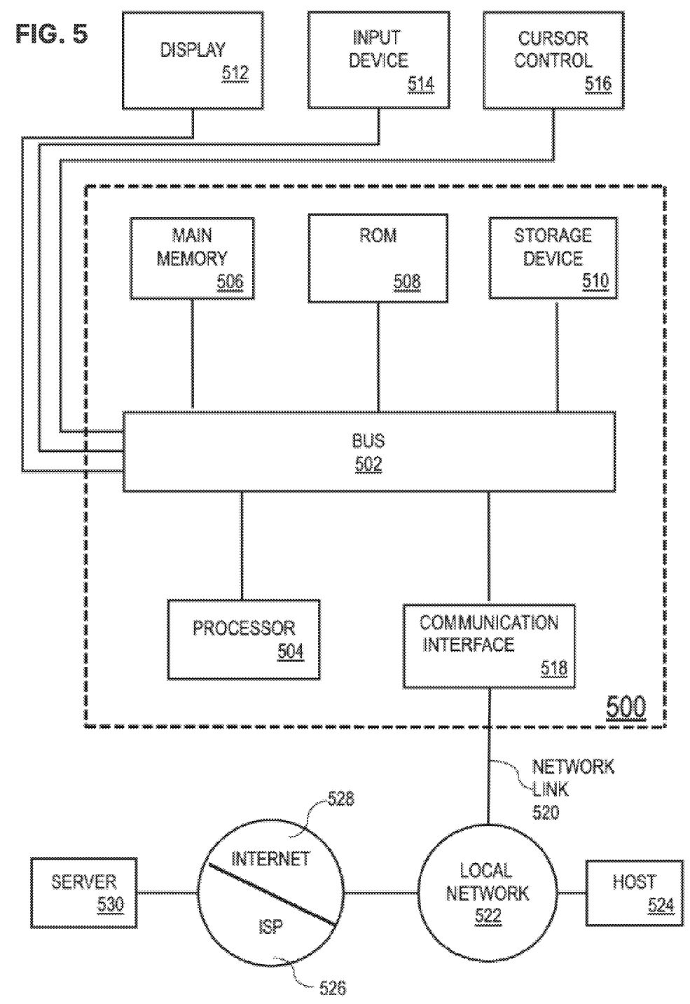

For example, FIG. 5 is a block diagram that illustrates a computer system 500 upon which an embodiment of the disclosure may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read-only memory (ROM) 508 or other static storage device coupled to bus 502 for storing static information and instructions for processor 504. A storage device 510, such as a magnetic disk, optical disk, or a Solid State Drive (SSD) is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or solid state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

### 12. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected, and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer readable media comprising instructions which, when executed by one or more hardware processors, cause performance of operations comprising:
accessing a set of patient data for a patient, the patient data identifying a first patient condition;
accessing a first clinical guideline of a plurality of clinical guidelines, wherein the first clinical guideline is associated with a first medical procedure associated with the first patient condition;
applying a first generative artificial intelligence (GenAI) model to the first clinical guideline to generate a first clinical guideline questionnaire and a first set of clinical guidelines rules;
applying a second GenAI model to the first clinical guideline questionnaire and a first subset of patient data of the set of patient data for the patient to populate the first clinical guideline questionnaire with the first subset of patient data for the patient to generate a first prefilled questionnaire;
applying a third GenAI model to the first prefilled questionnaire and the first set of clinical guideline rules to generate a determination if the first patient condition satisfies the first set of clinical guideline rules for medical necessity for the first medical procedure;
generating an evaluation indicating whether the first patient condition satisfies the first set of clinical guideline rules for medical necessity of the first medical procedure; and
displaying the evaluation in a user interface for review by a clinician.

2. The one or more non-transitory computer readable media of claim 1, wherein the operations further comprise:
providing a first prompt to the first GenAI model to generate a clinical guideline questionnaire for each clinical guideline of the plurality of clinical guidelines.

3. The one or more non-transitory computer readable media of claim 1, wherein the operations further comprise:
providing a third prompt to the third GenAI model to evaluate prefilled questionnaires and corresponding sets of clinical guideline rules to determine if patient conditions meet medical necessity for medical procedures.

4. The one or more non-transitory computer readable media of claim 1, wherein the third GenAI model provides explanations with the determination of medical necessity.

5. The one or more non-transitory computer readable media of claim 1, wherein the operations further comprise:
parsing the set of patient data into a plurality of subsets of patient data, the plurality of subsets of patient data including the first subset of patient data;
generating a first patient data vector embedding for the first subset of patient data;
generating a first question vector embedding for a first question of the first clinical guideline questionnaire;
calculating a first similarity score for the first patient data vector embedding and the first question vector embedding; and
determining that the first similarity score for the first patient data vector embedding and the first question vector embedding exceeds a threshold score for including the first subset of patient data as a potential answer to the first question.

6. The one or more non-transitory computer readable media of claim 5, wherein the operations further comprise:
generating a second patient data vector embeddings for a second subset of patient data;
calculating a second similarity score for the second patient data vector embedding and the first question vector embedding;
determining that the second similarity score is below the threshold score; and
in response to determining that the second similarity score is below the threshold score, refraining from providing the second subset of patient data to the second GenAI model.

7. The one or more non-transitory computer readable media of claim 1, wherein the operations further comprise:
generating a prior authorization request; and
submitting the prior authorization request to a third-party payer.

8. A method comprising:
accessing a set of patient data for a patient, the patient data identifying a first patient condition;
accessing a first clinical guideline of a plurality of clinical guidelines, wherein the first clinical guideline is associated with a first medical procedure associated with the first patient condition;
applying a first GenAI model to the first clinical guideline to generate a first clinical guideline questionnaire and a first set of clinical guidelines rules;
applying a second GenAI model to the first clinical guideline questionnaire and a first subset of patient data of the set of patient data for the patient to populate the first clinical guideline questionnaire with the first subset of patient data for the patient to generate a first prefilled questionnaire;
applying a third GenAI model to the first prefilled questionnaire and the first set of clinical guideline rules to generate a determination if the first patient condition satisfies the first set of clinical guideline rules for medical necessity for the first medical procedure;
generating an evaluation indicating whether the first patient condition satisfies the first set of clinical guideline rules for medical necessity of the first medical procedure; and
displaying the evaluation in a user interface for review by a clinician,
wherein the method is performed by at least one device including a hardware processor.

9. The method of claim 8, further comprising:
providing a first prompt to the first GenAI model to generate a clinical guideline questionnaire for each clinical guideline of the plurality of clinical guidelines.

10. The method of claim 8, further comprising:
providing a third prompt to the third GenAI model to evaluate prefilled questionnaires and corresponding sets of clinical guideline rules to determine if patient conditions meet medical necessity for medical procedures.

11. The method of claim 8, wherein the third GenAI model provides explanations with the determination of medical necessity.

12. The method of claim 8, further comprising:
parsing the set of patient data into a plurality of subsets of patient data, the plurality of subsets of patient data including the first subset of patient data;
generating a first patient data vector embedding for the first subset of patient data;
generating a first question vector embedding for a first question of the first clinical guideline questionnaire;
calculating a first similarity score for the first patient data vector embedding and the first question vector embedding; and
determining that the first similarity score for the first patient data vector embedding and the first question vector embedding exceeds a threshold score for including the first subset of patient data as a potential answer to the first question.

13. The method of claim 12, further comprising:
generating a second patient data vector embeddings for a second subset of patient data;
calculating a second similarity score for the second patient data vector embedding and the first question vector embedding;
determining that the second similarity score is below the threshold score; and
in response to determining that the second similarity score is below the threshold score, refraining from providing the second subset of patient data to the second GenAI model.

14. The method of claim 8, further comprising:
generating a prior authorization request; and
submitting the prior authorization request to a third-party payer.

15. A system comprising:
one or more hardware processors;
one or more non-transitory computer-readable media; and
program instructions stored on the one or more non-transitory computer-readable media that, when executed by the one or more hardware processors, cause the system to perform operations comprising:
accessing a set of patient data for a patient, the patient data identifying a first patient condition;
accessing a first clinical guideline of a plurality of clinical guidelines, wherein the first clinical guideline is associated with a first medical procedure associated with the first patient condition;
applying a first GenAI model to the first clinical guideline to generate a first clinical guideline questionnaire and a first set of clinical guidelines rules;
applying a second GenAI model to the first clinical guideline questionnaire and a first subset of patient data of the set of patient data for the patient to populate the first clinical guideline questionnaire with the first subset of patient data for the patient to generate a first prefilled questionnaire;
applying a third GenAI model to the first prefilled questionnaire and the first set of clinical guideline rules to generate a determination if the first patient condition satisfies the first set of clinical guideline rules for medical necessity for the first medical procedure;
generating an evaluation indicating whether the first patient condition satisfies the first set of clinical guideline rules for medical necessity of the first medical procedure; and
displaying the evaluation in a user interface for review by a clinician.

16. The system of claim 15, wherein the operations further comprise:
providing a first prompt to the first GenAI model to generate a clinical guideline questionnaire for each clinical guideline of the plurality of clinical guidelines.

17. The system of claim 15, wherein the operations further comprise:
providing a third prompt to the third GenAI model to evaluate prefilled questionnaires and corresponding sets of clinical guideline rules to determine if patient conditions meet medical necessity for medical procedures.

18. The system of claim 15, wherein the third GenAI model provides explanations with the determination of medical necessity.

19. The system of claim 15, wherein the operations further comprise:
parsing the set of patient data into a plurality of subsets of patient data, the plurality of subsets of patient data including the first subset of patient data;
generating a first patient data vector embedding for the first subset of patient data;
generating a first question vector embedding for a first question of the first clinical guideline questionnaire;
calculating a first similarity score for the first patient data vector embedding and the first question vector embedding; and
determining that the first similarity score for the first patient data vector embedding and the first question vector embedding exceeds a threshold score for including the first subset of patient data as a potential answer to the first question.

20. The system of claim 19, wherein the operations further comprise:
generating a second patient data vector embeddings for a second subset of patient data;
calculating a second similarity score for the second patient data vector embedding and the first question vector embedding;
determining that the second similarity score is below the threshold score; and
in response to determining that the second similarity score is below the threshold score, refraining from providing the second subset of patient data to the second GenAI model.
